(19) United States
(12) Patent Application Publication
(10) Pub. No.: US 2024/0252799 A1
(43) Pub. Date: Aug. 1, 2024

(54) WAVE AND PARTICLE BASED CELL TARGETING TREATMENTS

(71) Applicant: Applied Physics, Inc., New York, NY (US)

(72) Inventors: Gianni Martire, New York, NY (US); Ed Rietman, Grantham, NH (US)

(21) Appl. No.: 18/426,185

(22) Filed: Jan. 29, 2024

Related U.S. Application Data
(60) Provisional application No. 63/441,760, filed on Jan. 27, 2023.
(60) Provisional application No. 63/540,860, filed on Sep. 27, 2023.

(51) Int. Cl. A61M 37/00; A61N 5/06; A61N 5/067

(52) U.S. Cl. CPC A61M 37/0092; A61N 5/062; A61N 5/067

(57) ABSTRACT

Systems configured for treating invasive agent cells can include particles and a wave generation system. Particles can be introducible into a living being and can include surface features that facilitate attachment to invasive agent cells. The wave generation system can provide a specific wave pattern that can include a Fibonacci element and/or waves at different frequencies. The specific wave pattern can actuate the particles to damage or destroy invasive agent cells to which they are attached. The living being can be human and the invasive agent cells can be cancer cells. Waves can include sound, light, magnetic, or electromagnetic waves. The particles can be nanoparticles, such as functionalized gold nanoparticles having surface features that include citrate, lactate, glycol and/or biological components. Actuation can involve moving particles in response to the specific wave pattern in a manner that results in lysis of or immune response to the attached invasive agent cells.

### CROSS-REFERENCE TO RELATED APPLICATIONS

This application claims the benefit of U.S. Provisional Patent Application No. 63/441,760, filed Jan. 27, 2023, and titled “SOUND AND NANOPARTICLE BASED CELL TARGETING TREATMENTS,” which application is hereby incorporated by reference in its entirety herein. This application also claims the benefit of U.S. Provisional Patent Application No. 63/540,860, filed Sep. 27, 2023, and also titled “SOUND AND NANOPARTICLE BASED CELL TARGETING TREATMENTS.” which application is also hereby incorporated by reference in its entirety herein.

### TECHNICAL FIELD

The present disclosure relates generally to cell treatments, and more particularly to treatments involving the targeted destruction of invasive agent cells within living beings.

### BACKGROUND

Cancer is a disease caused by an uncontrolled division of abnormal cells within a living being and is the primary cause of mortality worldwide according to the World Health Organization. Traditional treatments for cancer and other deadly or harmful invasive agents within a living being include surgery, radiation, and chemotherapy, depending on the type of cancer or other invasive agent. While various forms of radiation and chemotherapy can be very good at destroying cancer cells and other invasive agents within a living being, particularly with respect to newly formed cells, these forms of treatment tend to be indiscriminate in their destruction. Unfortunately, this results in the destruction of desirable healthy cells within the body, such that many forms of radiation and chemotherapy must be tempered or augmented with alternative types of treatments so as not to destroy entire body parts or even kill a human patient or other living being. This can affect the overall efficacy of many such types of treatment.

While traditional ways of treating cancer cells and other invasive agents have worked well in the past, improvements are always helpful. In particular, what is desired are invasive agent treatment techniques and systems having improved accuracies in destroying cancerous cells and other invasive agents while preserving other living cells within a living being.

### SUMMARY

It is an advantage of the present disclosure to provide invasive agent cell treatment techniques and systems having improved accuracies in destroying cancerous cells or other invasive agents while preserving other living cells within a living being. The disclosed features, apparatuses, systems, and methods relate to targeting and damaging or destroying cancer cells or other invasive agents within a human or other living being. In particular, the disclosed systems and methods can involve targeting invasive agents within a living being using specific wave patterns, such as sound, light, magnetic, or electromagnetic waves. This can also include introducing particles into the living being, such as functionalized nanoparticles, and then actuating the nanoparticles with a specific wave pattern when the nanoparticles have attached to the cancer cells or other invasive agents.

In various embodiments of the present disclosure, systems configured to treat invasive cell agents can include a plurality of particles and a wave generation system. The plurality of particles can be configured for introduction into a living being and can include surface features configured to facilitate attachment of at least a portion of the plurality of particles to invasive agent cells within the living being. The wave generation system can be configured to provide a specific pattern of waves to the living being, the specific wave pattern including waves at one or more frequencies. The specific wave pattern can be configured to actuate the plurality of particles to damage, destroy, or create an immune response to the invasive agent cells to which they are attached.

In various detailed embodiments, the living being can be a human and the invasive agent cells can be cancer cells. The plurality of particles can include functionalized nanoparticles selected from the group consisting of gold, silver, liposomes, and dendrimers. The plurality of particles can be contained within a fluid configured for injection into the bloodstream of the living being. Actuating the plurality of particles can include inducing one or more movements, rotations, or changes in temperature, shape, confirmation, or chemical structure in at least a portion of the plurality of particles in response to the specific wave pattern. Also, actuating the plurality of particles can result in lysis of or immune response to invasive agent cells to which the at least a portion of the plurality of particles are attached. The specific wave pattern can include pulses of waves interspersed between pulses of no waves, and wherein durations of the pulses vary to form ratios between the pulses of waves and the pulses of no waves. The relationship of the ratios can form an arithmetic, geometric, or Fibonacci sequence. In various arrangements, the waves can include sound waves. The waves can also include light waves, magnetic waves, or electromagnetic waves.

In further embodiments of the present disclosure, various methods of treating an invasive agent within a living being are provided. Pertinent process steps can include providing a plurality of particles, introducing the plurality of particles into the living being, facilitating attachment of a significant amount of the plurality of particles to invasive agent cells within the living being, and directing a specific wave pattern toward the living being. The plurality of particles can include surface features configured to facilitate attachment of a significant amount of the plurality of particles to invasive agent cells within the living being. The specific wave pattern can include waves at one or more frequencies and can be configured to actuate the plurality of particles to damage, destroy, or create an immune response to the invasive agent cells to which they are attached.

In various detailed embodiments, the specific wave pattern can include pulses of waves interspersed between pulses of no waves. Durations of the pulses can vary to form ratios between the pulses of waves and the pulses of no waves. The relationship of the ratios can form an arithmetic, geometric, or Fibonacci sequence. In addition, the pulse pattern may be from a quasiperiodic oscillator such as a Duffing oscillator, Lorenz attractor, or Henon attractor. An additional process step can include actuating the plurality of particles automatically with the specific wave pattern. The actuating can result in damaging, destroying, or provoking an immunological response to invasive agent cells to which any of the plurality of particles are attached. Actuating the plurality of particles can involve rotational or translational movement of the particles due to the specific wave pattern. In some arrangements, the waves can include sound waves and the plurality of particles can include functionalized nanoparticles.

In still further embodiments of the present disclosure, systems configured to treat cancer within a human can include at least a wave generation system configured to project a specific wave pattern at the human. The specific wave pattern can include waves at one or more frequencies and can be configured to induce lysis of or immune response to a significant amount of cancer cells within the human. The specific wave pattern can include pulses of waves interspersed between pulses of no waves. Duration of the pulses can vary to form ratios between the pulses of waves and the pulses of no waves, and the relationship of the ratios of the pulses can form an arithmetic, geometric, Fibonacci sequence, or quasiperiodic function. In some arrangements, the specific wave pattern can include a specific sound pattern having a Fibonacci word element, wherein the Fibonacci word element includes a series of 0 and 1 values that each represent pulses of no sound and pulses of sound respectively, and wherein each of the 0 and 1 pulses have the same time length. In some arrangements, the system can also include a plurality of functionalized nanoparticles configured for introduction into the human. The plurality of functionalized nanoparticles can include surface features configured to facilitate attachment of the functionalized nanoparticles to cancer cells within the human without also facilitating attachment of the functionalized nanoparticles to a significant amount of healthy cells of the human. The functionalized nanoparticles can be configured to be actuated by the specific wave pattern.

In various further embodiments of the present disclosure, systems configured for treating invasive agent cells can include at least a plurality of nanoparticles and a sound generation system. The plurality of nanoparticles can be configured for introduction into a living being and can include surface features configured to facilitate attachment of a significant amount of the plurality of nanoparticles to invasive agent cells within the living being without also facilitating attachment of a significant amount of the plurality of nanoparticles to healthy cells of the living being. The sound generation system can be configured to provide a specific sound pattern to the living being and the specific sound pattern can include sound waves at one or more sound frequencies. The specific sound pattern can be configured to actuate the plurality of nanoparticles to damage, destroy, or create an immune response to the invasive agent cells to which they are attached.

In various detailed embodiments, the living being can be a human and the invasive agent cells can be cancer cells. The plurality of nanoparticles can include functionalized nanoparticles selected from the group consisting of gold, silver, liposomes, and dendrimers. In some arrangements, the plurality of nanoparticles can be contained within a fluid configured for injection into the bloodstream of the living being. The nanoparticle surface features can include one or more citrate, lactate, glycol, or norbornene components, polymeric strings having a multiple of such components in a repeating pattern, or any combination thereof. Alternatively, or in addition, the nanoparticle surface features can include one or more biological components, such as antibodies or other proteins. Actuating the plurality of nanoparticles can include inducing one or more movements, rotations, or changes in temperature, shape, confirmation, or chemical structure in each of at least a portion of the plurality of nanoparticles in response to the specific sound pattern. The actuating can result in lysis of or immune response to invasive agent cells to which the at least a portion of the plurality of nanoparticles are attached. In some arrangements, the specific sound pattern can include pulses of infrasound or ultrasound interspersed between pulses of no sound, sound at audible or non-audible frequencies, or both. The lengths of the pulses of infrasound or ultrasound can vary to form ratios between the pulses of infrasound or ultrasound. The relationship of the ratios of the pulses of sound can form an arithmetic, geometric, or Fibonacci sequence, among other possible sequences, such as quasiperiodic signals.

In further embodiments of the present disclosure, various methods of treating an invasive agent within a living being are provided. Pertinent process steps can include providing a plurality of nanoparticles, introducing the nanoparticles into the living being, facilitating attachment of the nanoparticles to invasive agent cells, and directing a specific sound pattern toward the living being. The plurality of nanoparticles can include surface features configured to facilitate attachment of a significant amount of the plurality of nanoparticles to invasive agent cells within the living being without also facilitating attachment of a significant amount of the plurality of nanoparticles to healthy cells of the living being. Facilitating attachment can involve attachment of a significant amount of the plurality of nanoparticles to invasive agent cells. The specific sound pattern can include sound waves at infrasound or ultrasound frequencies and can be configured to actuate the plurality of nanoparticles to damage or destroy invasive agent cells to which they are attached.

In various detailed embodiments, facilitating attachment can include allowing a sufficient amount of time for the significant amount of the plurality of nanoparticles to attach to the invasive agent cells. Also, introducing the nanoparticles can involve injecting into the living being a fluid containing the plurality of nanoparticles. The specific sound pattern can include pulses of infrasound or ultrasound interspersed between pulses of no sound, sound at audible or non-audible frequencies, or both, and the lengths of the pulses of infrasound can vary to form ratios between the pulses of sound. The relationship of the ratios of the pulses of sound can form an arithmetic, geometric, or Fibonacci sequence, among other possible patterns. An additional process step can include actuating the plurality of nanoparticles automatically with the specific sound pattern. The actuating can result in damaging, destroying, or provoking an immunological response to invasive agent cells to which any of the plurality of nanoparticles are attached. In some arrangements, actuating the plurality of nanoparticles can involve rotational or translational movement of the nanoparticles due to the specific sound pattern, among other possible movements.

In various further embodiments of the present disclosure, a system configured for treating living cells can include at least a sound generation system configured to project a specific sound pattern. The specific sound pattern can include sound waves at infrasound or ultrasound frequencies, and the specific sound pattern can be configured to induce lysis of or immune response to a significant amount of living cells within a living being.

In various detailed embodiments, the specific sound pattern can include pulses of sound interspersed between pulses of no sound, and the lengths of the pulses of sound can vary to form ratios between the pulses of sound. The relationship of the ratios of the pulses of sound can form an arithmetic, geometric, Fibonacci sequence, or other quasiperiodic signal. In some arrangements, the specific sound pattern can include a Fibonacci word element within the specific sound pattern, and this Fibonacci word element can include a series of 0 and 1 values that each represent pulses of no sound and pulses of sound respectively. Each of the 0 and 1 pulses can have the same time length. The Fibonacci word element within the specific sound pattern can be the overwhelming or only cause of inducing the lysis of or the immune response to the significant amount of living cells. The specific sound pattern may also not cause any significant lysis of or immune response to any other cells within the living being besides the invasive cells. The system can also include a plurality of functionalized nanoparticles configured for introduction into the living being and these functionalized nanoparticles can include surface features configured to facilitate attachment of the functionalized nanoparticles to invasive cells within the living being without also facilitating attachment of the functionalized nanoparticles to a significant amount of healthy cells of the living being. The functionalized nanoparticles can be configured to be actuated by the specific sound pattern.

Other apparatuses, methods, features, and advantages of the disclosure will be or will become apparent to one with skill in the art upon examination of the following figures and detailed description. It is intended that all such additional apparatuses, methods, features and advantages be included within this description, be within the scope of the disclosure, and be protected by the accompanying claims.

### BRIEF DESCRIPTION OF THE DRAWINGS

The included drawings are for illustrative purposes and serve only to provide examples of possible structures, arrangements, and methods for providing sound and nanoparticle based cell targeting treatments. These drawings in no way limit any changes in form and detail that may be made to the disclosure by one skilled in the art without departing from the spirit and scope of the disclosure.

FIG. 1 illustrates in block diagram format an example invasive agent cell treatment system according to one embodiment of the present disclosure.

FIG. 2 illustrates a flowchart of an example summary method of treating an invasive agent within a living being according to one embodiment of the present disclosure.

FIG. 3A illustrates in block diagram format an example arrangement of functionalized nanoparticles and normal and invasive agent cells within a living being according to one embodiment of the present disclosure.

FIG. 3B illustrates in block diagram format the arrangement of FIG. 3A with the functionalized nanoparticles attached to an invasive agent cell according to one embodiment of the present disclosure.

FIG. 3C illustrates in block diagram format the arrangement of FIG. 3B with the functionalized nanoparticles rotating while attached to the invasive agent cell according to one embodiment of the present disclosure.

FIG. 4 illustrates a flowchart of an example detailed method of treating cancer cells within a human using sound to actuate injected functionalized nanoparticles according to one embodiment of the present disclosure.

FIG. 5A illustrates a graph of an example combination of sound sine waves at ultrasonic and infrasonic frequencies according to one embodiment of the present disclosure.

FIG. 5B illustrates a graph of an alternative example combination of sound square waves at ultrasonic and infrasonic frequencies according to one embodiment of the present disclosure.

FIG. 6A illustrates a graph of an example pulsed infrasound treatment pattern at 2 Hz provided in a Fibonacci sequence according to one embodiment of the present disclosure.

FIG. 6B illustrates a graph of an alternative example pulsed infrasound treatment pattern at 10 Hz provided in a Fibonacci sequence according to one embodiment of the present disclosure.

FIG. 7 illustrates in top plan view an example test chamber and sound generation system processor configured to test different eukaryotic model organisms for selective targeting and destruction according to one embodiment of the present disclosure.

FIG. 8A illustrates a magnified picture of an example set of algae cells prior to treatment according to one embodiment of the present disclosure.

FIG. 8B illustrates a magnified picture of an example set of yeast cells prior to treatment according to one embodiment of the present disclosure.

FIG. 8C illustrates a magnified picture of an example combined set of algae cells and yeast cells prior to treatment according to one embodiment of the present disclosure.

FIG. 8D illustrates a magnified picture of an example combined set of algae and yeast cells after a first treatment process according to one embodiment of the present disclosure.

FIG. 8E illustrates a magnified picture of an example combined set of algae and yeast cells after a second treatment process according to one embodiment of the present disclosure.

FIG. 9A illustrates a graph of an example sound wave pattern having a Fibonacci word component according to one embodiment of the present disclosure.

FIG. 9B illustrates a graph of a comparable sound wave pattern having no Fibonacci word component according to one embodiment of the present disclosure.

FIG. 10 illustrates in top plan view an example test chamber and transducer arrangement configured to be coupled to a sound generation system for testing different model organisms for selective targeting and destruction according to one embodiment of the present disclosure.

FIG. 11A illustrates charts of testing results on yeast cells using sound waves without nanoparticles according to one embodiment of the present disclosure.

FIG. 11B illustrates charts of testing results on Haematococcus algae cells using sound waves without nanoparticles according to one embodiment of the present disclosure.

FIG. 11C illustrates charts of testing results on Chlorella algae cells using sound waves without nanoparticles according to one embodiment of the present disclosure.

FIG. 12 illustrates a chart of an example Fibonacci binary string according to one embodiment of the present disclosure.

FIG. 13A illustrates in front perspective view an example alternative testing chamber and transducer arrangement configured to be coupled to a sound generation system for testing different model organisms for selective targeting and destruction according to one embodiment of the present disclosure.

FIG. 13B illustrates in bottom perspective view the alternative testing arrangement of FIG. 12A according to one embodiment of the present disclosure.

FIG. 14 illustrates charts of further testing results on Haematococcus algae cells using sound waves without nanoparticles according to one embodiment of the present disclosure.

FIG. 15A illustrates a magnified picture of an example combined set of algae cells and yeast cells after a treatment process involving a sound wave pattern having a Fibonacci word component according to one embodiment of the present disclosure.

FIG. 15B illustrates a magnified picture of an example combined set of algae cells and yeast cells after a treatment process involving a comparable sound wave pattern having no Fibonacci word component according to one embodiment of the present disclosure.

FIG. 16 illustrates charts of testing results and associated error bars for tests on cells of different sizes using sound waves without nanoparticles according to one embodiment of the present disclosure.

FIG. 17 illustrates charts of testing results on chlorella cells without nanoparticles using sound waves having Fibonacci sound patterns at different frequencies according to one embodiment of the present disclosure.

FIG. 18 illustrates charts of testing results on yeast cells without nanoparticles using sound waves having Fibonacci sound patterns at different frequencies according to one embodiment of the present disclosure.

FIG. 19 illustrates charts of testing results on Haematococcus algae cells without nanoparticles using sound waves having Fibonacci sound patterns at different frequencies according to one embodiment of the present disclosure.

FIG. 20 illustrates charts of testing results on chlorella cells without nanoparticles using a continuous wave laser and a pulsed laser having a Fibonacci pulse pattern according to one embodiment of the present disclosure.

FIG. 21 illustrates charts of endocytosis testing and recovery results on chlorella cells using gold and silver nanoparticles according to one embodiment of the present disclosure.

FIG. 22 illustrates charts of testing results on chlorella cells ingested with gold and silver nanoparticles and treated using sound waves having Fibonacci sound patterns at a set frequency according to one embodiment of the present disclosure.

FIG. 23 illustrates charts of testing results on Haematococcus algae cells ingested with gold and silver nanoparticles and treated using sound waves having Fibonacci sound patterns at a set frequency according to one embodiment of the present disclosure.

### DETAILED DESCRIPTION

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

The present disclosure relates in various embodiments to features, apparatuses, systems, and methods for the treatment of invasive agents in living beings, such as the targeted destruction of cancer cells within humans. This can generally involve targeting and destroying or at least damaging cancer or other invasive agent cells while avoiding significant damage to all or most of the healthy and beneficial cells within the living being. Such targeting and damaging can be facilitated through the use of particles. Such particles can include functionalized nanoparticles that can be actuated or activated by way of specific wave patterns. Such specific wave patterns can include sound patterns, which can include audible, non-audible, infrasound, ultrasound, sound harmonics, or any combination thereof. Actuation of the particles can occur when they are attached to invasive agents, cancer cells, or other cells or items of interest, whereupon the particles can then be configured to react to a trigger to damage or destroy the invasive agents, cancer cells and/or other items of interest.

In various embodiments of the present disclosure, novel sound patterns can be used in creating infrasonic and/or ultrasonic vibrations that are configured to harm or destroy targeted invasive cells or bodies. The specific sound patterns can provide a trigger that vibrates certain materials at certain sound frequencies. The vibrated materials can include functionalized nanoparticles that are specifically engineered to attach to cancer cells and/or other targets and also to vibrate, rotate, or otherwise move in response to the specific sound patterns. Such nanoparticle movement can then result in damaging or destroying the cancer cells, invasive agents, and/or other items of interest. In some arrangements, the provided sound patterns can follow an arithmetic, geometric, or Fibonacci sequence, or some other advantageous pattern, such as a quasiperiodic oscillator.

Although various embodiments disclosed herein discuss the treatment of cancer cells within humans using nanoparticles and infrasound patterns, it will be readily appreciated that the disclosed features, apparatuses, systems, and methods can also be used to treat other invasive cells or agents within any form of living being. Similarly, any other suitable substitute or alternative materials that take advantage of the disclosed features can be used to achieve the same or similar results in these or similar applications.

In addition, the disclosed systems and methods can be used to target items other than cells or living beings. For example, the disclosed wave patterns can be used to target inanimate objects and materials in order to break down or destroy such objects and materials. This can include rocks, earth, glass, and other simple materials, for example, as well as complex or composite materials or structures. The various specialized sound, vibrational, or other wave patterns disclosed herein can then be used to focus or isolate on a particular type of material or object, such that the material or object of interest is targeted and broken down or destroyed without also breaking down or destroying other materials or objects near the targeted item.

Furthermore, while various embodiments disclosed herein discuss the use of sound waves and wave patterns, it will be readily appreciated that the disclosed features, apparatuses, systems, and methods can be used with other types of waves and wave patterns. These can include the use of, for example, vibrational waves, light waves, magnetic waves, and electromagnetic waves, among other types of waves. It will be understood that all examples disclosed herein that discuss the use of sound waves and wave patterns can use any of these other types of vibrations, waves, and wave patterns as an alternative to or in addition to sound waves, where suitable.

In addition, while various embodiments disclosed herein discuss the use of nanoparticles, which can include functionalized nanoparticles, it will be readily appreciated that the disclosed features, apparatuses, systems, and methods can be used with other types of particles, which can similarly include functionalized particles. It will be understood that all examples disclosed herein that discuss the use of nanoparticles and functionalized nanoparticles can use any other suitable types of particles as an alternative to or in addition to nanoparticles and functionalized nanoparticles.

Furthermore, while infrasound is often cited herein as a particular type of sound used for treatment, it will be understood that ultrasound, audible sound, non-audible sound, and any other type of sound can be used alternatively or in addition to infrasound in some applications. Also, while nanoparticle and sound patterns are used in combination for some of the illustrative treatments provided herein for purposes of illustration, it will be understood that nanoparticles or other particles are not necessary for all treatments, and that specific sound or other wave patterns alone may be used in some cases. Other applications, arrangements, and extrapolations beyond the illustrated embodiments are also contemplated.

As is generally well known, nanotechnology can refer to the development and manufacture of materials at the atomic and molecular nanoscale level at an order of magnitude smaller than about 100 nm. Nanoparticles have specific properties that come from their small volumes associated with extensive surface areas, which can allow for advantageous properties and changes in thermal behavior, resistance, solubility, conductivity, and catalytic activity, among other properties. The use of nanoparticles, particularly metal nanoparticles, has expanded in therapeutics due to their unique properties of small size, large surface area to volume ratio, high reactivity to living cells, stability over high temperatures and translocation into cells.

Nanoparticles (and other suitably small particles) are available in different sizes and shapes due to their ability to react and combine with other nanoparticles in their surroundings. Useful nanoparticle base materials include gold, silver, titanium dioxide, iron, polymeric, lyposomal, and dendrimeric, among others. In particular, the inert and low cytotoxicity of gold results in gold being a particularly useful base material for nanoparticles when it comes to targeted delivery to and actions within biological tissues and systems within living beings. Sub-micron scale titanium dioxide particles may also prove viable for the various nanoparticle biomedical applications disclosed herein. Combining these base nanoparticles with various surface features can then result in functionalized nanoparticles suitable for use in the various systems and methods disclosed herein. Such surface features can include geometrical physical arrangements and biomolecules such as ligands, antibodies, and other proteins.

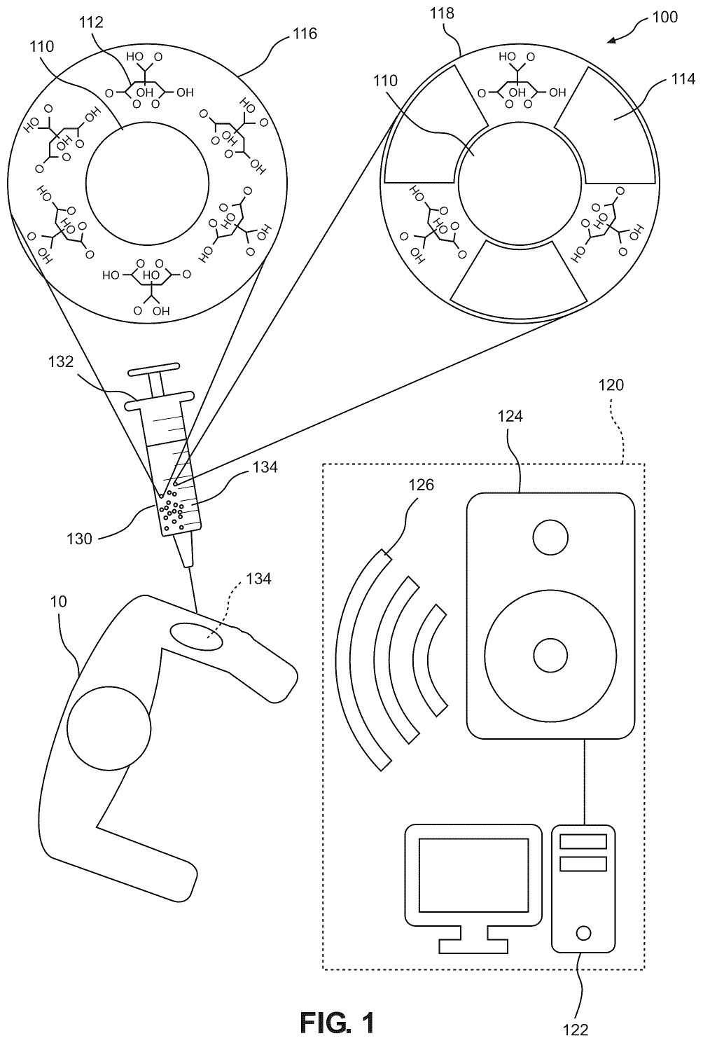

Referring first to FIG. 1, an example invasive agent cell treatment system is illustrated in block diagram format. Invasive agent cell treatment system 100 can be used to treat invasive agent cells, such as leukemia and other cancer cells, for example, within living beings, such as humans. In various embodiments, invasive agent cell treatment system 100 can include at least a plurality of nanoparticles 110 and a sound generation system 120. Nanoparticles 110 can be functionalized by way of various surface features, which can include geometric surface features 112, biological surface features 114, or both. Other types of surface features are also possible. A simple functionalized nanoparticle 116 can be formed from a base nanoparticle 110 with one type of surface feature 112, while a compound functionalized nanoparticle 118 can be formed from a base nanoparticle 110 having multiple different types of surface features 112, 114, for example. Sound generation system 120 can include one or more processing components 122 and one or more speakers 124 configured to produce sound waves 126 toward a target, such as, for example, a human patient 10. At least a portion of sound waves 126 can form a specific sound pattern that is configured to trigger or actuate functionalized nanoparticles 116, 118, and these can include sound waves at infrasonic frequencies, ultrasonic frequencies, audible frequencies, non-audible frequencies, or any combination thereof.

In various embodiments, invasive cell treatment system 100 can also include a delivery component 130 configured to introduce the plurality of nanoparticles 110 into the human 10. In some arrangements this delivery component 130 can include, for example, a syringe 132 filled with a fluid 134 containing multiple functionalized nanoparticles, which can include simple functionalized nanoparticles 116, compound functionalized nanoparticles 118, or both. As will be readily appreciated, syringe 132 can be configured to inject fluid 134 into the bloodstream of human patient 10. Other types of delivery components are also possible, and these can include delivery components that are ingestible, inhalable, transdermal, or any other suitable way of introducing nanoparticles into a human or other living being. In general, nanoparticles 110 and their delivery media need not form a medication or other prescriptive component, but rather can form an effectively innate serum, fluid, or other composition suitable for effectively introducing the nanoparticles into the human or other living being.

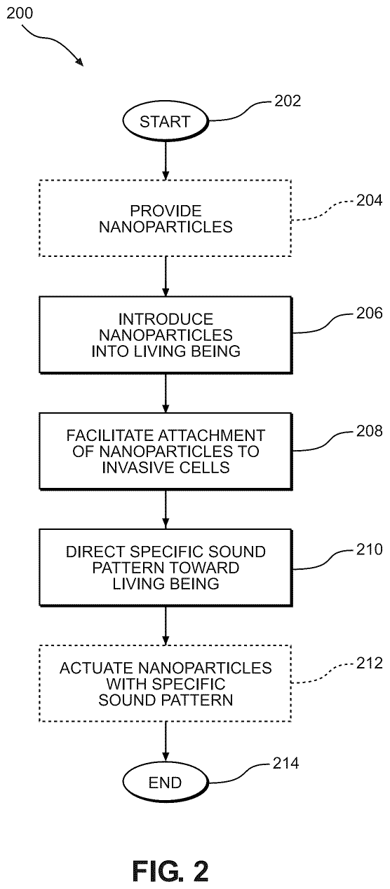

Moving next to FIG. 2, a flowchart of an example summary method 200 of treating an invasive agent within a living being is provided. Summary method 200 can represent a broad overview of treating an invasive agent, and it will be understood that various other steps, features, and details of such a broad overview method are not provided here for purposes of simplicity. After a start step 202, an optional first process step 204 can involve providing nanoparticles. This can involve the designing, engineering, and forming of functionalized nanoparticles through a variety of ways, for example, or can simply involve procuring a suitable number of nanoparticles for use in the treatment process.

As a following process step 206, the nanoparticles can be introduced into a living being. The living being can be a human patient, for example, and the introduction can be by way of any suitable delivery component. Such introduction can involve injection, ingestion, inhalation, dermal exposure, or any other suitable introduction technique, as will be readily appreciated by one of skill in the art.

At the next process step 208, attachment of the nanoparticles to invasive agent cells within the living being can be facilitated. This can involve simply allowing an adequate amount of time to pass for the nanoparticles to circulate through the blood or tissue of the living being to allow sufficient exposure to the invasive agent cells. Various features or procedures can also be used to accelerate this process, such as the addition of one or more medications or other agents to stimulate circulation and/or attraction of the nanoparticles to the invasive agent cells.

At a subsequent process step 210, a specific sound pattern can be directed toward the living being. The specific sound pattern can include one or more infrasound components, one or more ultrasound components, one or more audible components, one or more non-audible components, or any combination thereof, and can be designed to elicit a reaction in a substantial amount or all of the nanoparticles. Various features and characteristics of possible specific sound patterns are provided in greater detail below.

At a following optional process step 212, some or all of the nanoparticles can be actuated by way of the specific sound pattern. This can be performed automatically as a result of directing the specific sound pattern toward the living being, such as by the nanoparticles inherently reacting to the vibrations of the provided sound waves. In some arrangements, this process step 212 can be considered an automatic result of directing the specific sound pattern. Actuation of some or all of the nanoparticles can then result in reactions of those nanoparticles that in turn damages or destroys a significant amount of the invasive agent cells within the living being. The method can then end at end step 214.

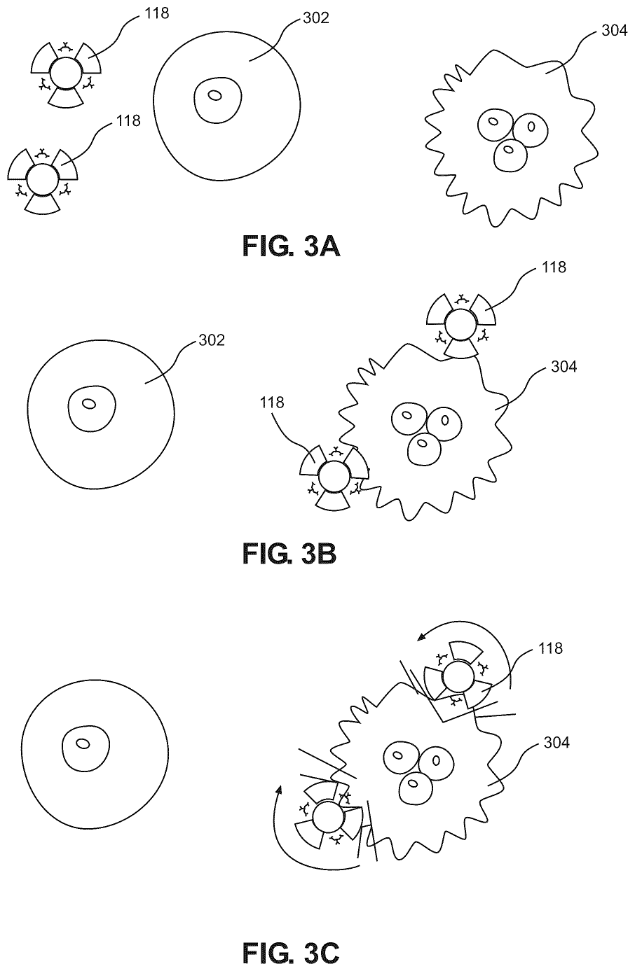

Turning now to FIGS. 3A through 3C, example arrangements of functionalized nanoparticles and normal and invasive agent cells within a living being are shown in block diagram format. FIG. 3A depicts two compound functionalized nanoparticles 118, a single normal cell 302, and a single invasive agent cell 304, which can be a cancer cell, for example. It will be readily appreciated that there can be many multiples of each of the functionalized nanoparticles 118 and cells 302, 304, and that other types of nanoparticles and cells may also be present within the living being, which can be a human. As depicted in FIG. 3A, the functionalized nanoparticles 118 can be newly introduced and unattached to any other item within the human or other living being.

In FIG. 3B, the functionalized nanoparticles 118 are depicted as having traveled through the living being, ignoring the normal cell 302, and becoming attached to the invasive agent cell 304. Such attachment can be facilitated by one or more surface features on the functionalized nanoparticles 118, which surface features can be designed to ignore normal or healthy cells while attaching the nanoparticles to invasive agent cells, which again can be cancer cells. These surface features can include, for example, geometric surface features, biological surface features, other surface features, or any combination thereof.

In FIG. 3C, the functionalized nanoparticles 118 are depicted as being actuated, which again can occur due to the presence of a specific sound pattern being imparted on the human or other living being and reaching the nanoparticles. Actuation can occur while the functionalized nanoparticles 118 are attached to the invasive agent cell 304. Such actuation can involve vibration or other movement of the functionalized nanoparticles 118. For example, some functionalized nanoparticles 118 can be configured to rotate while attached to the invasive agent cell in response to the specific sound pattern, which rotation can cause tearing or other damage to the invasive agent cell 304. Such tearing or other damage can occur due to sharp spikes or other surface features on the nanoparticles and can result in lysis of the invasive agent cell. Other forms of nanoparticle motion and resulting damage to the invasive agent cell are also possible. For example, some nanoparticles may simple vibrate sufficiently enough to cause damage to or even destroy cancer cells or other invasive agent cells to which they are attached.

In some arrangements, functionalized nanoparticles can be engineered to attach to invasive agent cells and/or to elicit a response from other items of interest besides cancer cells or invasive agent cells or bodies. For example, some embodiments can involve mimicking cancer cells or other invasive agent cells, or bind an irritating agent to the invasive cells so as to elicit a response from the immune system of the living being, which can provoke an immune response intrinsically or when activated using sound, and at such time direct the immune system to recognized the invasive agent cells. As another example, some embodiments can involve targeting cancer cells or invasive agent cells indirectly, such as through the use of white blood cells within the living being. In such situations, functionalized nanoparticles can have biological surface features that directly target white blood cells, with the knowledge that the white blood cells will naturally target and attach to the invasive agents within the living being. Actuation of the functionalized nanoparticles can then result in the destruction or damaging of the directly attached white blood cells and also the cancer cells or invasive agent cells to which the white blood cells are attached. Other targeting arrangements using specialized functionalized nanoparticles are also possible.

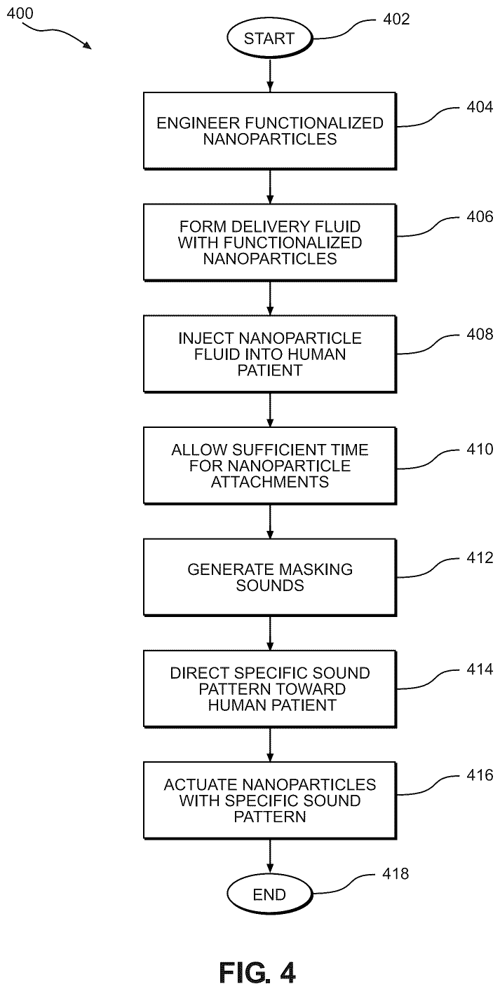

Moving next to FIG. 4, a flowchart of an example detailed method 400 of treating cancer cells within a human using sound to actuate injected functionalized nanoparticles is provided. Again, detailed method 400 can represent one possible way of treating cancer cells, and it will be understood that various other steps, features, and details of such a detailed method are not provided here for purposes of simplicity. After a start step 402, a first process step 404 can involve engineering functionalized nanoparticles for use in treatment of a specific cancer. This can involve designing and forming compound functionalized nanoparticles that are specifically configured to attach to invasive agent cells while ignoring normal or healthy cells, and that are also configured to react to specific sound patterns once they are so attached.

At a following process step 406, a delivery fluid containing the functionalized nanoparticles can be formed. Such a delivery fluid can be relatively inert and may simply provide a vehicle through which the functionalized nanoparticles can be delivered into a human patient. Process step 408 can then involve injecting the nanoparticle fluid into the human patient. This can be done by way of a typical syringe and injection arrangement. Again, injection of a fluid containing functionalized nanoparticles can be just one way of delivering the nanoparticles into a human, and alternative delivery components and methods are also possible. For example, functionalized nanoparticles can be placed into a capsule or solid pill media and can then be ingested by the human patient in some arrangements.

At subsequent process step 410, a sufficient amount of time can be allowed to lapse for the functionalized nanoparticles to target and attach to various cancer cells within the human patient. Such amounts of time may vary for a variety of factors and can be slowed or accelerated by one or more other items or processes, as will be readily appreciated. Various surface features of the functionalized nanoparticles can be configured to facilitate attachment of a significant amount of the nanoparticles to cancer cells (or other invasive agent cells) within the human patient without also facilitating attachment of a significant amount of the nanoparticles to healthy cells of the human.

At the next process step 412, one or more masking sounds can be generated. Such masking sounds can be provided to the human patient to mask, drown out, or otherwise downplay any uncomfortable effects of a separate specific sound pattern used to actuate the functionalized nanoparticles. This can be desirable due to the ugly or disconcerting nature of a sound pattern that is intended to irritate or lyse target cells. Examples of such masking sounds can include sounds of nature or one or more musical songs, among other possible masking sounds. As a particular example, where a therapeutic specific sound pattern is expected to last about five minutes, then a popular music song of at or about that same length can be played for the human patient at a higher volume than the volume of the specific sound pattern.

At following process step 414, a specific sound pattern can be directed toward the human patient. Again, the specific sound pattern can include one or more infrasound components, one or more ultrasound components, one or more audible components, one or more non-audible components, or any combination thereof, and can be designed to elicit a reaction in a substantial amount or all of the nanoparticles. Various components of the specific sound pattern can form harmonics between them to facilitate or amplify the triggering or actuation of the functionalized nanoparticles.

At a subsequent process step 416, the functionalized nanoparticles can then be actuated with the specific sound pattern. As noted above, this can take place automatically due to the specific formation of the functionalized nanoparticles and the exact arrangement of the specific sound pattern. Actuation can result in movement and/or other actions by the functionalized nanoparticles while attached to the cancer cells. In some arrangements, such movement can merely be vibrations, while in other arrangements such movement can also include rotation. Other movements can include pulling, poking, or tearing at the cancer cells or other invasive agent cells. In some arrangements, the functionalized nanoparticles can even be configured to fully or partially explode, resulting in shrapnel bits. Some or all of these various forms of motion can serve to destroy or significantly damage the cancer cells. The method can then end at end step 418.

For the foregoing methods 200 and 400, it will be appreciated that not all process steps are necessary, and that other process steps may be added in some arrangements. Furthermore, the order of steps may be altered in some cases, and some steps may be performed simultaneously. For example, step 406 may be performed sooner in the process in some arrangements, while process steps 412 through 416 may be performed simultaneously. Although known process steps are provided for the various techniques in method 400, it will be appreciated that any other suitable similar method for treating cancer cells can also be used. Other variations and extrapolations of the disclosed methods will also be readily appreciated by those of skill in the art.

Various additional details, extrapolations, and/or alternatives can be applied to the systems, features, and methods disclosed above. For example, some nanoparticles can involve a base particle having a shape that can be changed upon attachment and/or actuation in some arrangements. Alternatively, or in addition, various surface features on the functionalized nanoparticles can also have shapes or arrangements that may be changed. In some embodiments, portions of some or all nanoparticles can be formed from Nitinol or other shape memory materials that can move or change shapes due to triggers other than specific sound patterns.

As noted above, some of the surface features on the disclosed functionalized nanoparticles can include protein structures that are configured to find and attach to cancer cells, other invasive cells or bodies of interest, and/or other targeted items, such as white blood cells. Such protein structures can include platelets, ligands, antibodies, and other proteins structures, for example. Multiple protein structures can be formed as surface features on a given nanoparticle, such that a single nanoparticle can possibly attach to multiple invasive agent cells. In addition, multiple protein structures can increase the odds that a given nanoparticle will eventually bind to an invasive agent cell or other cell or item of interest.

In some arrangements, various functionalized nanoparticles can include protein structures or other surface features that allow for attachment to different types of invasive agent cells. Such nanoparticles can be considered as multiple function nanoparticles. For example, a given functionalized nanoparticle can contain various different protein structures along its surface that facilitate attachment to different strains of flu viruses. Using a plurality of such multiple function functionalized nanoparticles can then allow for the targeted attachment and destruction of multiple different types of invasive agents. In situations where a human patient has a flu virus but the exact strain of virus is unknown, then multiple functional nanoparticles having protein structures specific to all of the most common flu viruses for a given flu season can be used to treat that human patient. Similarly, introduction of such nanoparticles can be used as an immunizing agent to cover many different flu strains at once. Other applications and further extrapolations of the systems and methods disclosed herein are also possible.

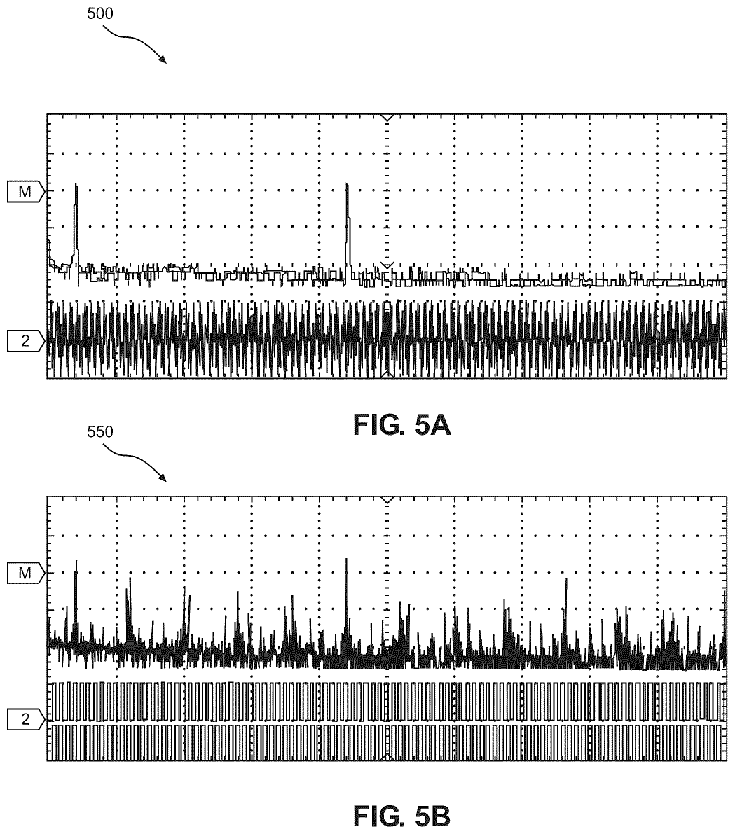

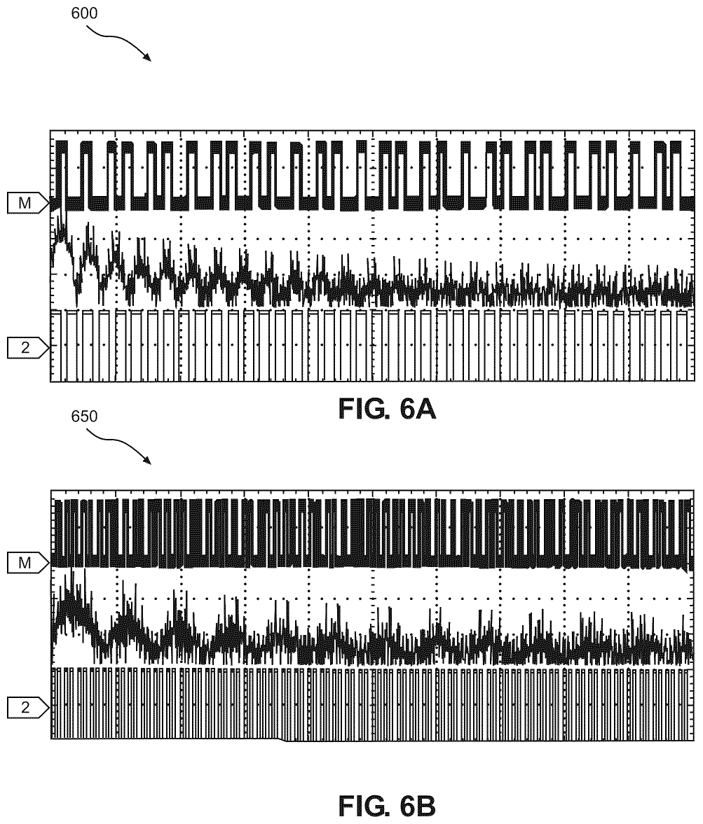

Transitioning to FIGS. 5A through 6B, various graphs of sound waves are provided. FIG. 5A illustrates a graph of an example combination 500 of sound sine waves at ultrasonic and infrasonic frequencies, while FIG. 5B depicts a graph of an alternative example combination 550 of sound square waves at ultrasonic and infrasonic frequencies. Either of combination 500 or combination 550 can provide a basis for a specific sound pattern as utilized above in system 100, although further sound waves and some modifications are also possible.

In various embodiments, the specific sound pattern can be a pulsed pattern of sound. As such, sound can be provided at a given frequency for certain pulsed amounts of time with alternating amounts of time between the pulsed sounds. The alternating amounts of time can be filled with no sound or with sound at inert or noninteractive frequencies. The certain pulsed amounts of time for sounds at frequencies of interest can vary from one pulse to the next and can form relative ratios with respect to one another. In various embodiments, a pattern of the relative amounts or ratios of pulsed sounds of interest can follow an arithmetic, geometric, or Fibonacci sequence, among other possible patterns. FIG. 6A illustrates a graph 600 of an example pulsed infrasound treatment pattern at 2 Hz provided in a Fibonacci sequence, while FIG. 6B depicts a graph 650 of an alternative example pulsed infrasound treatment pattern at 10 Hz provided in a Fibonacci sequence.

In the case of a Fibonacci sequence, for example, a first pulse length can be set at 1 second, a second pulse length can be set at 1 second, a third pulse length can be set at 2 seconds, a fourth length at 3 seconds, a fifth length at 5 seconds, and a sixth length at 8 seconds. Further pulse lengths, if used, can then be set at lengths of 13, 21, 34 seconds, and so forth. Other pulse lengths and starting amounts can also be used, such as, for example, a starting pulse length of 0.1 seconds. Other starting points in the Fibonacci sequence may also be used. Other types of arithmetic or geometric sequences may also be used.

Alternatively, or in addition, the amount of infrasound or other sound frequencies can vary according to a pattern. Again, in the case of a Fibonacci sequence, for example, a first pulse of sound can be provided at 1 Hz, a second pulse can be at 1 Hz, a third pulse can be at 2 Hz, and subsequent pulses can be at 3, 5, 8, 13 Hz, and so forth. Other starting frequencies can be used, and other sequences such as arithmetic or geometric sequences may also be used. As noted above, other types of wave patterns can be delivered alternatively or in addition to sound wave patterns, and these can include light waves, magnetic waves, electromagnetic waves, or any other suitable type of wave.

While the foregoing examples provide a pattern of pulses that can follow a Fibonacci, arithmetic, geometric, or other type of sequence, it is also contemplated that other components of sound waves or other waves can alternatively or also be delivered in such a pattern. For example, the frequency, amplitude, or other suitable component of the sound waves or other waves can be varied and delivered according to such a pattern. In some cases this can be done in place of a pattern of pulses, while in other cases there can be multiple patterns simultaneously. For example, a pattern of sound pulses can follow a Fibonacci, arithmetic, geometric, or other type of sequence, and each pulse within that pattern can have a variable frequency, a variable amplitude, or both, with such variations also following a Fibonacci, arithmetic, geometric, or other type of sequence. Various combinations can result in greater enhanced effects on the target invasive cells.

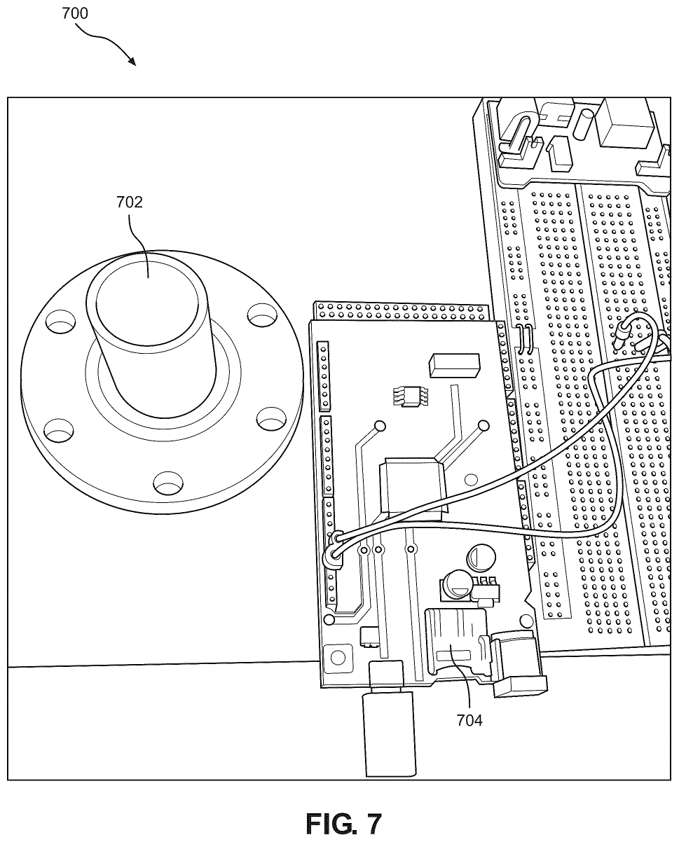

FIG. 7 illustrates in top plan view an example test chamber and sound generation system processor configured to test different eukaryotic model organisms for selective targeting and destruction. Testing arrangement 700 can include a test chamber 702, one or more speakers or other sound emitting devices (not shown) and a sound generation system processor 704. In various tests using testing arrangement 700, different types of eukaryotic model organisms can be placed into the test chamber 702 for tests using nanoparticles and other tests without the use of nanoparticles. All types of testing can include the implementation of sound waves of different frequencies and/or pulse patterns. The different types of eukaryotic model organisms can roughly extrapolate to the relative cell differences between cancer cells and healthy cells for some types of cancer.

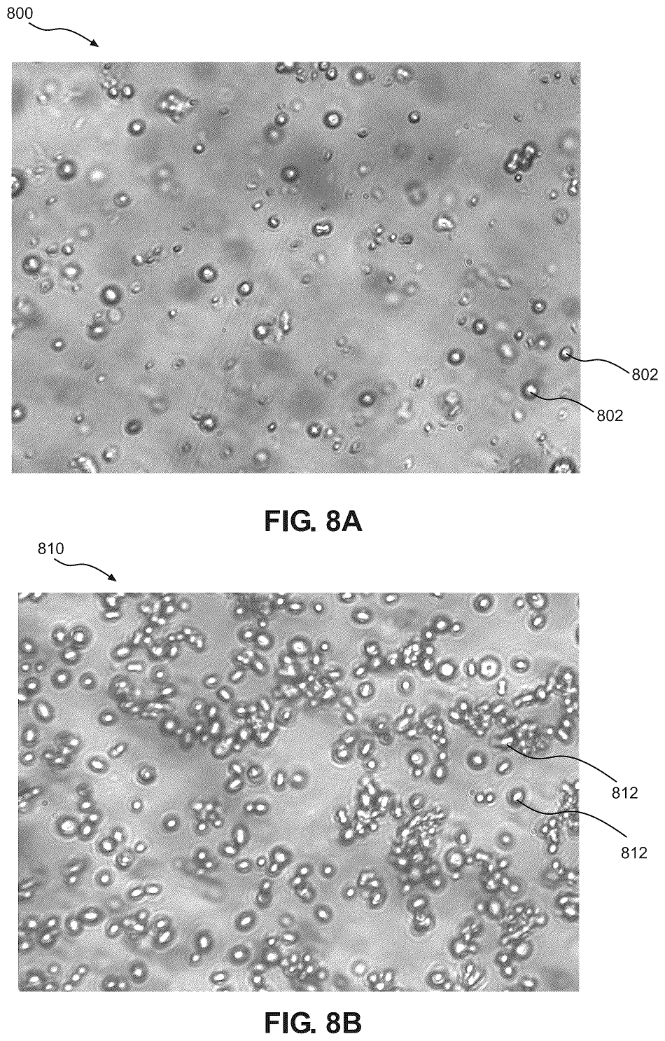

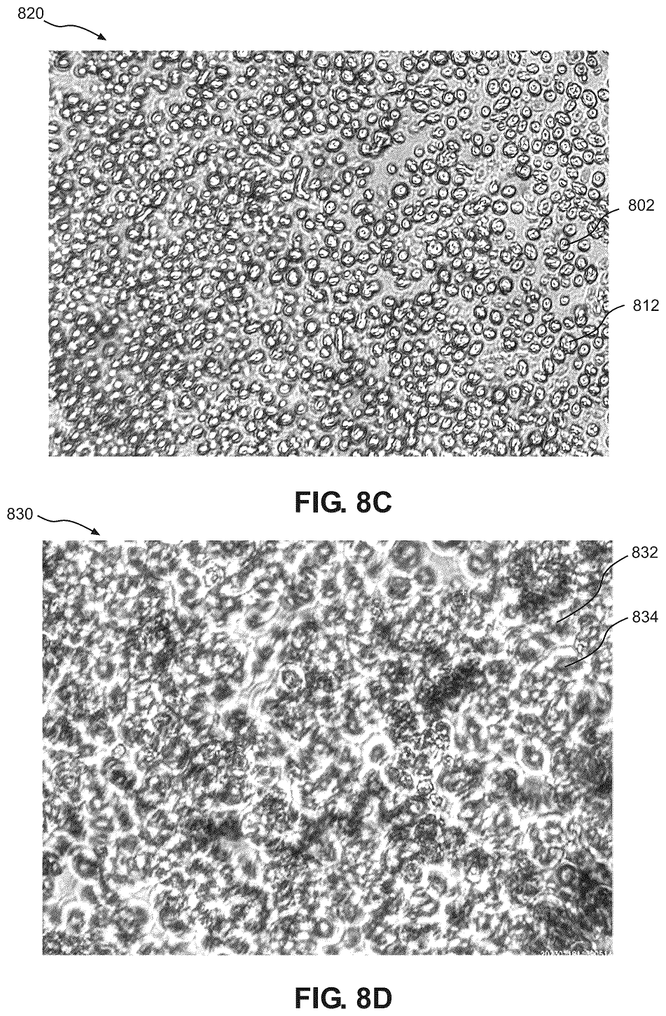

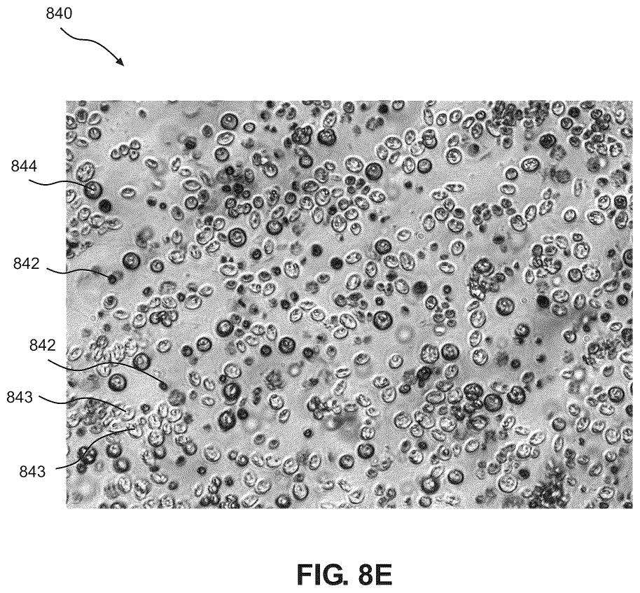

In one test arrangement, yeast (S. cerevisiae) was used as a first organism and a specific form of algae (nannochloropsis) was used as a second organism. The yeast used had 16 chromosomes (12 M DNA letters), whereas the selected algae had 32 chromosomes (29.3M DNA letters). These two organisms were tested together under various types of sound patterns without the use of nanoparticles in a first series of tests. Results are provided with respect to FIGS. 8A-E below.

FIG. 8A provides a magnified picture 800 of an example set of algae cells prior to treatment. Live algae cells 802 were about 10 microns in size and were contained within an inert fluid in a plastic petri dish. FIG. 8B illustrates a magnified picture 810 of an example set of yeast cells prior to treatment. Live yeast cells 812 were also about 10 microns in size and contained within an inert fluid in a plastic petri dish. FIG. 8C illustrates a magnified picture 820 of an example combined set of algae cells and yeast cells prior to treatment. Live algae cells 802 and live yeast cells 812 were both about 10 microns in size and contained within the same inert fluid in a plastic petri dish.

This combined arrangement of same sized algae and yeast cells as shown in magnified picture 820 were then subjected together to different treatment processes. FIG. 8D illustrates a magnified picture 830 of an example combined set of algae and yeast cells after a first treatment process. The first treatment process involved subjecting all cells to constant sound at about 25 Hz with no sound pattern for a period of about 10 minutes. As shown, virtually all cells were destroyed by this first treatment process, including dead algae cells 832 and dead yeast cells 834. FIG. 8E illustrates a magnified picture 840 of an example combined set of algae and yeast cells after a second treatment process. Given the mixed results shown in magnified picture 840, the cells shown were stained with methylene blue to aid in determining cell mortality. The second treatment process here involved subjecting all cells to sound waves at about 25 Hz having a pulsed pattern at a Fibonacci sequence with a 44 millisecond delay for an overall period of 10 minutes. Illustrated results include dead yeast cells 842, live yeast cells 843, and live algae cells 844. The dead yeast cells 842 appeared to have collapsed like deflated balloons. It was observed that the algae cells 844 were predominantly unaffected by the second treatment process, while a substantial number of the yeast cells 842 were killed by the second treatment process applied for a period of 10 minutes. Extrapolating these results, it is expected that modifying the sound pattern and overall length of this second treatment process should result in substantially all yeast cells being killed with substantially all algae cells remaining alive.

In various embodiments, the use of nanoparticles may not always be necessary to treat some cells effectively, such that specific sound patterns alone can be used. For example, certain environments where invasive cells are within a living being having normal or healthy cells can involve treatments that target the invasive cells with specific sound patterns only that significantly affect the invasive cells while leaving the normal or healthy cells intact or largely unaffected. Such arrangements can omit the nanoparticles 110 and the delivery component 130 of invasive agent cell treatment system 100 set forth above, such that they include only sound generation system 120 of the invasive agent cell treatment system.

In some embodiments, this can involve using sound patterns at specific frequencies that include binary Fibonacci strings known as “Fibonacci words.” In such arrangements, subjecting an overall environment of combined biological cells to these specific sound patterns can result in exploding or fragmenting cancer or other invasive cells while leaving other cells intact or largely unaffected. This can then flood the overall environment with cancer or invasive cell parts that include DNA, RNA, proteins, and the like, which molecular parts can then be recognized by a natural immune system as foreign or dangerous elements. Natural reactions of the immune system can then trigger an immune system response, such as the release or activation of natural killer cells, dendritic cells, T-cells, and the like. The rupture or fragmentation of cancer or other invasive cells can also result in the release of cytokines, which are proteins that can communicate with other cells to amplify an overall immune system response.

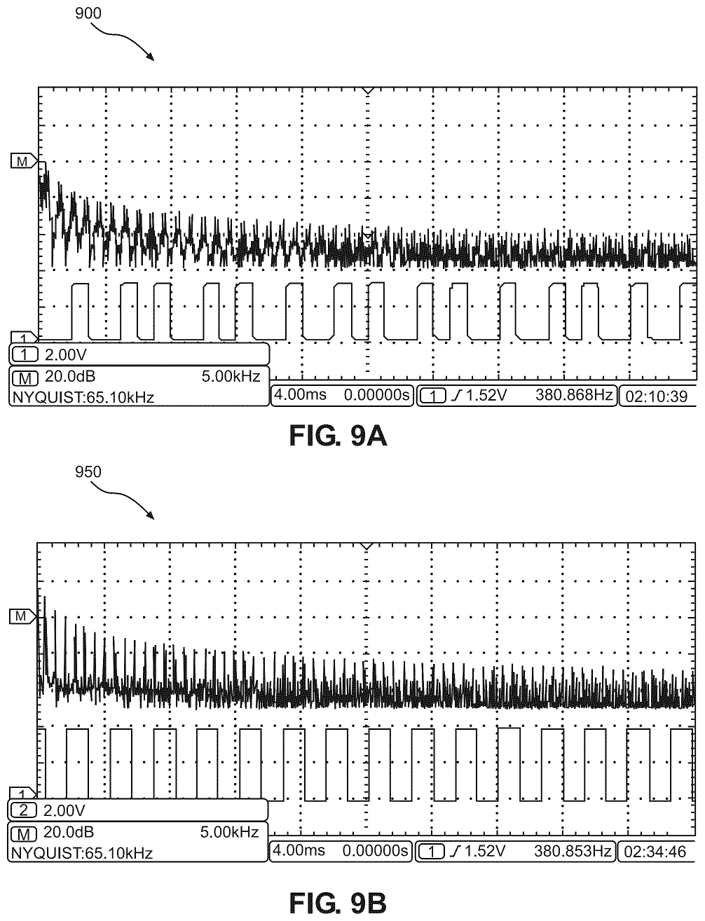

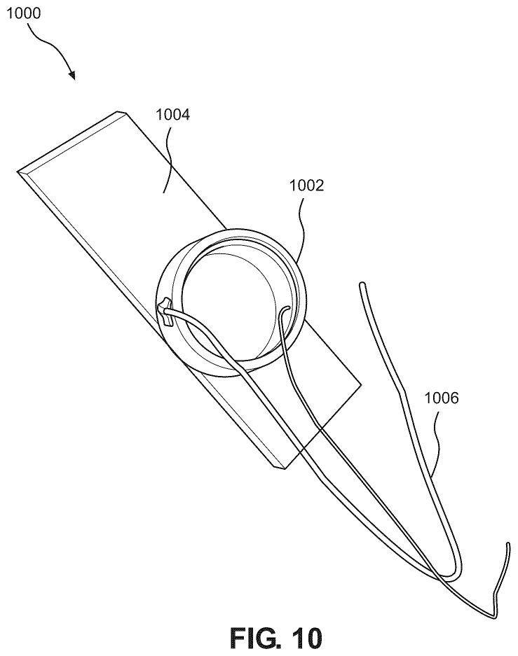

Transitioning now to FIGS. 9A and 9B, graphs are provided of example sound wave patterns having a Fibonacci word component and no Fibonacci word component respectively. Sound wave pattern 900 in FIG. 9A and sound wave pattern 950 in FIG. 9B both reflect sound wave patterns at about 380 Hertz and decay from a maximum amplitude of about 20 decibels. Sound wave pattern 900 includes a binary Fibonacci signal in its fast Fourier transform that is quasiperiodic and fractal-like in nature, such that its resonance peaks are blunted and not extreme. Conversely, sound wave pattern 950 is based on a simple square wave and has no binary Fibonacci signal component, such that its resonance peaks are sharp and extreme in nature. These sharp resonance peaks in sound wave pattern 950 tend to induce acoustophoresis that squeezes or otherwise impacts all cells in an affected environment regardless of cell size.

FIG. 10 illustrates in top plan view an example test chamber and transducer arrangement configured to be coupled to a sound generation system for testing different model organisms for selective targeting and destruction. Testing arrangement 1000 can include a test chamber 1002, a piezoelectric transducer epoxied to a glass slide 1004, and electrical wires 1006 configured to be driven by an amplifier (not shown) that is coupled to a sound generation system processor (not shown). In some arrangements, the processor can be configured to generate a signal of a sound pattern having a binary Fibonacci word component. In various tests using testing arrangement 1000, different types of eukaryotic model organisms and/or other biological cells can be placed into test chamber 1002 for tests using nanoparticles and other tests without the use of nanoparticles. All types of testing can include the implementation of sound waves of different frequencies and/or pulse patterns. The different types of eukaryotic model organisms and/or other biological cells can roughly extrapolate to the relative cell differences between cancer cells and healthy cells for some types of cancer.

In various tests within testing arrangement 1000, yeast (S. cerevisiae) at a size of about 5-10 microns was used as one cell organism, the algae Haematococcus pluvialis at a size of about 10-30 was used as another cell organism, and the algae Chlorella vulgaris at a size of about 2-5 microns was used as another cell organism. The size variations in these tests allow for investigating effects on different size cells, similar to what might be expected in different types of cancers or other invasive cells, as well as other biological cells of different sizes. For example, leukemia cells are about 10-20 microns in diameter, while red blood cells are about 6-8 microns and white blood cells are about 8-12 microns.

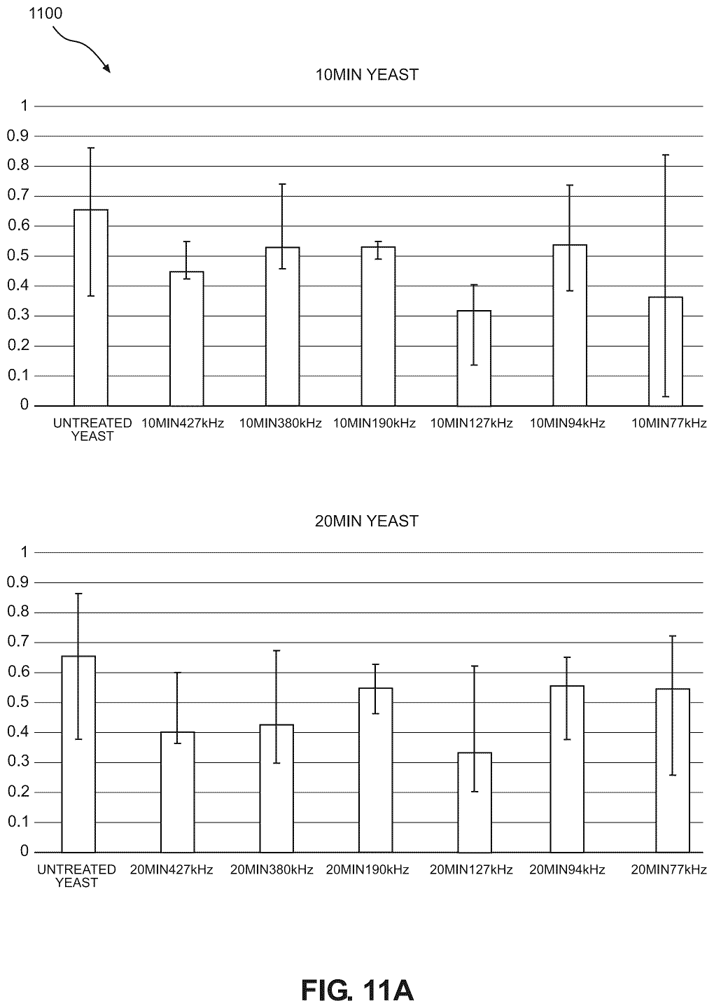

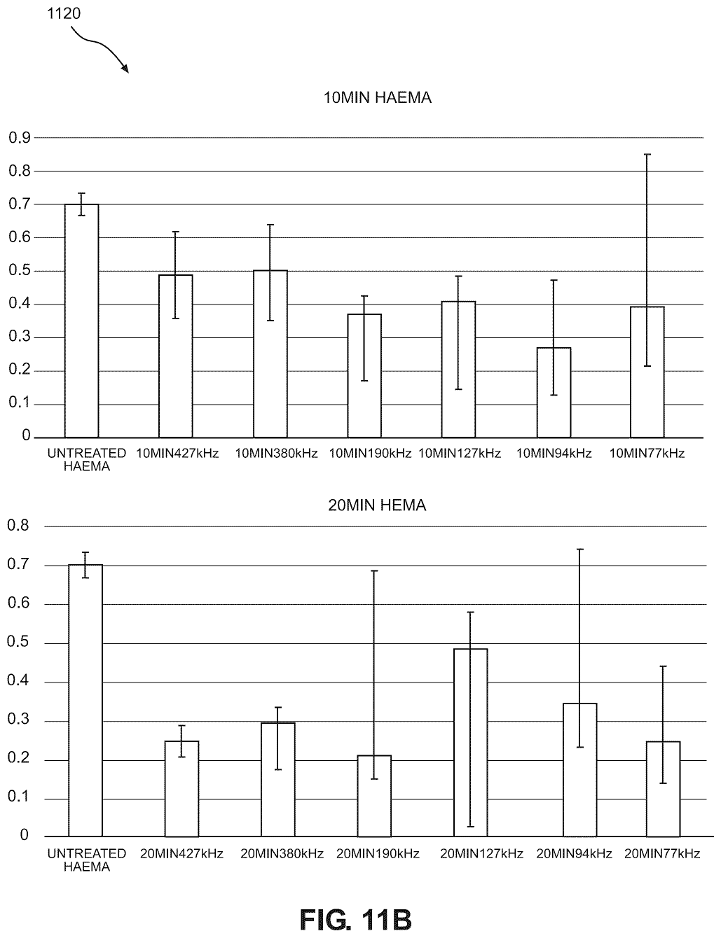

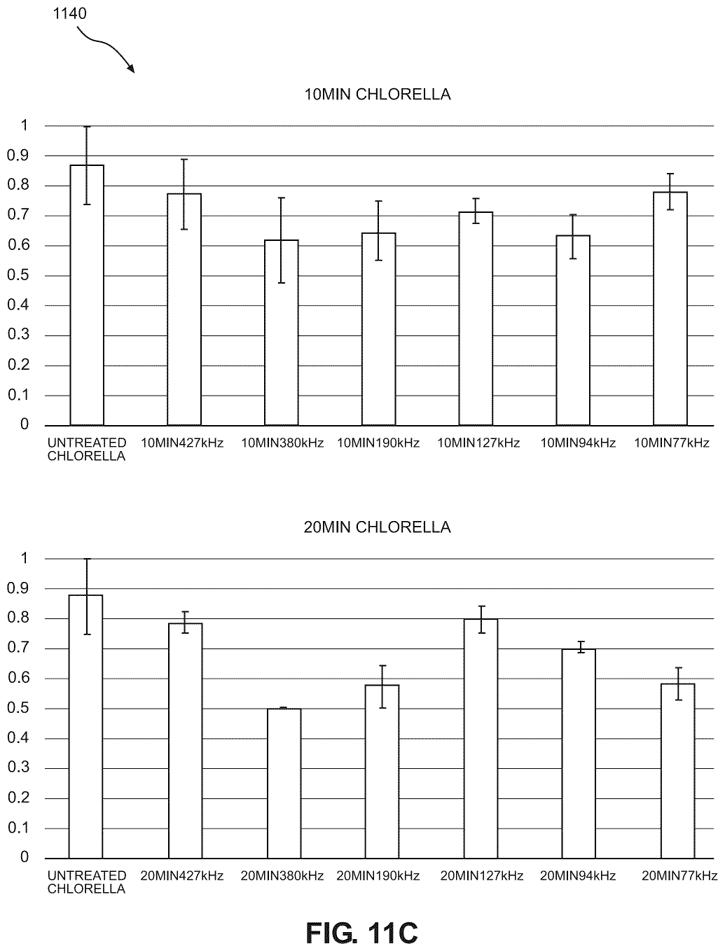

Results of the foregoing tests are reflected in FIGS. 11A-11C, which provide charts of testing results using sound waves without nanoparticles on yeast cells, Haematococcus algae cells, and Chlorella algae cells respectively. Each of charts 1100 for yeast, 1120 for Haematococcus, and 1140 for Chlorella include bar heights that represent the fraction of remaining live cells to the total number of original cells, expressed as a median of four separate tests. Bars in the top half of each chart reflect testing conducted for 10 minutes, while bars in the bottom half of each chart reflect testing for 20 minutes. Each bar represents testing at a different sound frequency, as labeled. Error bars at the top of each bar indicate the range observed over all tests for a given cell type, sound pattern frequency, and duration, as indicated. Given the results of these various tests, it can be seen that it is possible to use different sound pattern frequencies to differentiate between different sized cells. For example, the most cell destruction of yeast occurred at sound pattern frequencies of 127 Hz, while the most cell destruction of Haematococcus occurred at sound pattern frequencies of 94 Hz and the most cell destruction of Chlorella occurred at sound pattern frequencies of 380 Hz.

These results show that favorable outcomes are possible by using sound wave patterns without also implementing nanoparticles in some arrangements. In particular, it is possible to differentiate between cells of different sizes by varying the parameters of sound wave patterns directed at an environment of different cells. Specifically, it is possible to distinguish between the predominant destruction of yeast cells (5-10 microns) at certain sound patterns emitted at 127 Hz, the predominant destruction of Chlorella cells (2-5 microns) at the same sound patterns emitted at 380 Hz, and the predominant destruction of Haematococcus cells (10-20 microns) at the same sound patterns emitted at 94 Hz. This trend demonstrates that smaller sized cells are more susceptible to higher frequency sound patterns, while intermediate sized cells are more susceptible to intermediate frequency sound patterns and larger sized cells are more susceptible to lower frequency sound patterns. Further variations in sound pattern parameters can also be used to fine tune these distinctions between different cells.

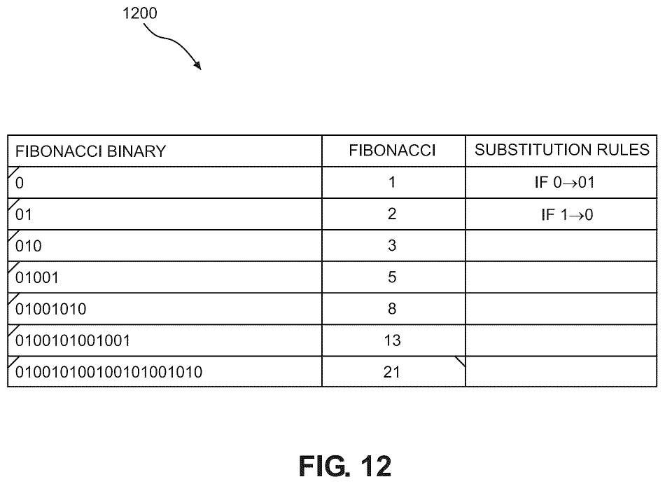

FIG. 12 illustrates a chart of an example Fibonacci binary string according to one embodiment of the present disclosure. As shown in chart 1200, a Fibonacci binary string can include binary values that correspond to base 10 values of a Fibonacci sequence. When converting the base 10 values from the middle column of chart 1200 to the binary string values of the left column of the chart, this corresponding string of “0” and “1” values can then be represented by pulses and rest periods in a sound pattern. For example, each “0” value of a string can represent a rest period in a sound pattern, while each “1” of a string can represent a period where sound vibration is delivered. In some embodiments, each numerical value in a given binary string can correspond to the same time increment of sound waves (1) or no sound waves (0) being delivered. For example, where each numerical value corresponds to a time period of 10 ms, then a Fibonacci binary string for “8” can be implemented by providing no sound for 10 ms, then sound for 10 ms, then no sound for 20 ms, then sound for 10 ms, then no sound for 10 ms, then sound for 10 ms, and then finally no sound for the last 10 ms. Other time period increments and other Fibonacci values are also possible under such an application.

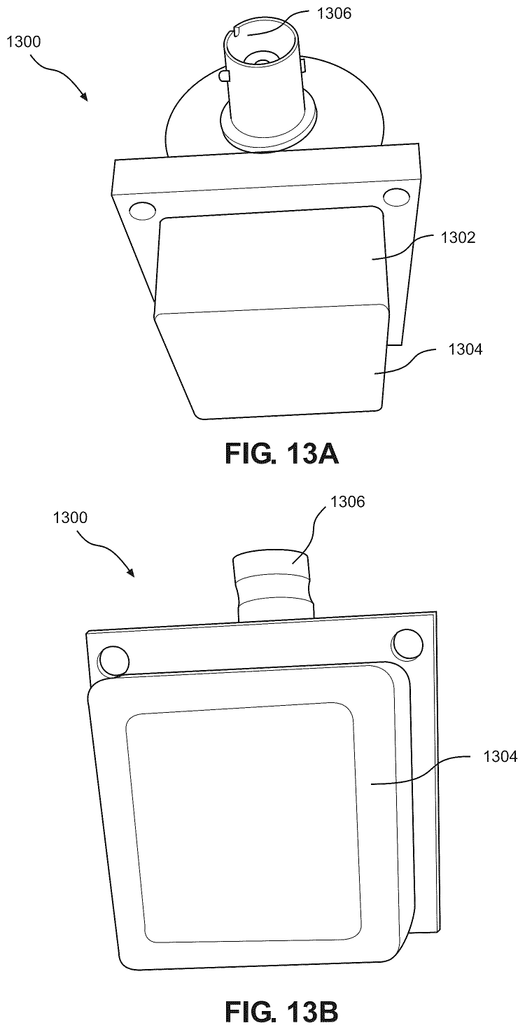

FIGS. 13A and 13B illustrate an example alternative testing chamber and transducer arrangement configured to be coupled to a sound generation system for testing different model organisms for selective targeting and destruction in front perspective and bottom perspective views respectively. Arrangement 1300 can include a testing chamber 1302 that can hold cells to be tested using a patterned sound wave application. Testing chamber 1302 can include a transducer 1304 along a bottom surface and a coupling component 1306 configured to deliver sound waves from an outside source.

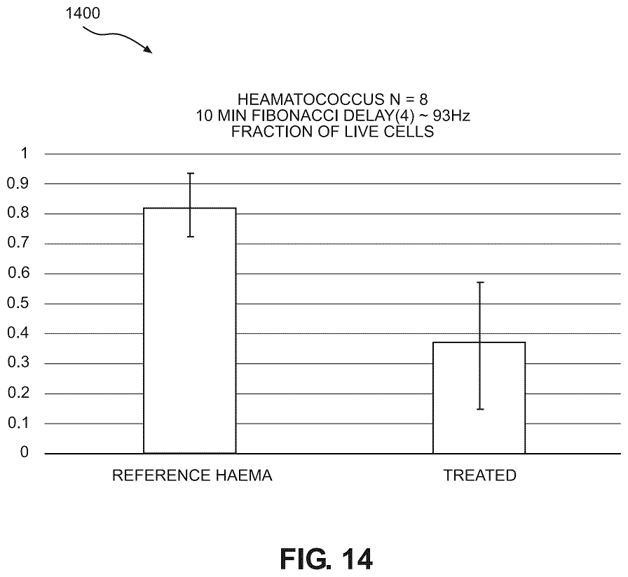

FIG. 14 illustrates charts of further testing results on Haematococcus algae cells using sound waves without nanoparticles. As in the foregoing results, each test result can include bar heights that represent the fraction of remaining live cells to the total number of original cells. In chart 1400, these are expressed as a median of eight separate tests for each set of cells. Testing for this series of results included sound waves delivered at about 93 Hz for a total time period of about 10 minutes per test. In the reference Haematococcus algae cells on the left, about 80% of the cells survived testing using sound patterns that did not include a Fibonacci binary word component. In the reference Haematococcus algae cells on the right, however, less than about 40% of the cells survived testing using sound patterns that did include a Fibonacci binary word component.

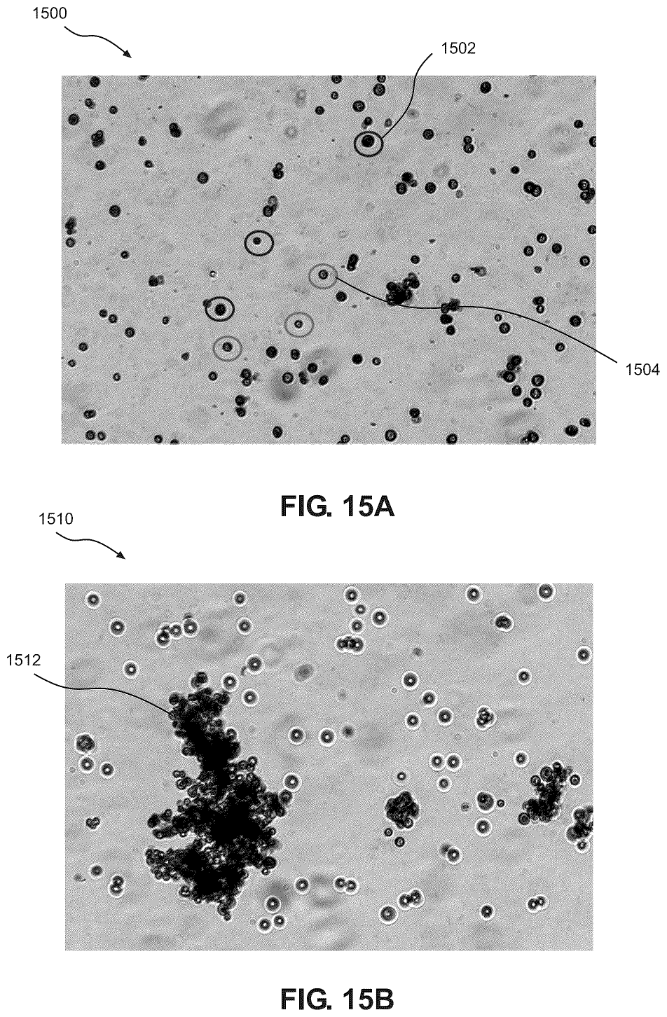

FIG. 15A illustrates a magnified picture of an example combined set of algae cells and yeast cells after a treatment process involving a sound wave pattern having a Fibonacci word component. As shown in magnified picture 1500, algae cells 1502 were largely destroyed while yeast cells 1504 mostly survived using a sound wave pattern at a given frequency delivered over a set time length, where the sound wave pattern had a Fibonacci word component.

FIG. 15B illustrates a magnified picture of an example combined set of algae cells and yeast cells after a treatment process involving a comparable sound wave pattern having no Fibonacci word component. As shown in magnified picture 1510, algae cells and yeast cells tended to bunch together into large clumps 1512 where no discernable pattern can be discernable between living and dead cells of either type when using a sound wave pattern at the same given frequency delivered over the same set time length in the foregoing example, except here where the sound wave pattern did not have a Fibonacci word component.

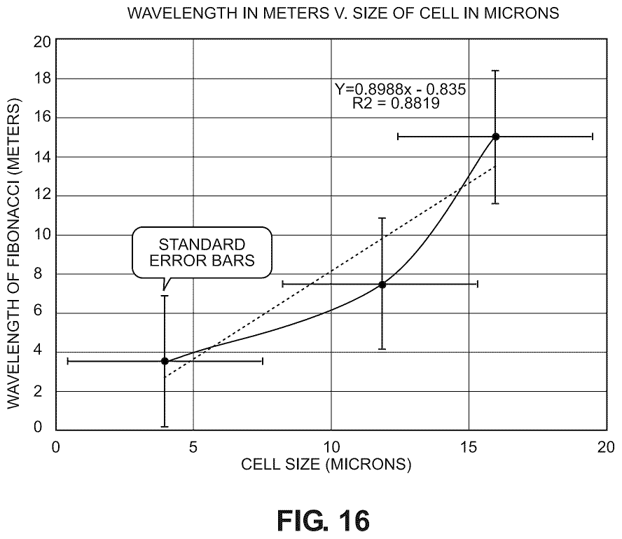

FIG. 16 illustrates charts of testing results and associated error bars for tests on cells of different sizes using sound waves without nanoparticles according to one embodiment of the present disclosure. Charted results are shown for chlorella cells having an average size of about 3.5 microns and subjected to sound patterns at a frequency of 380 Hz, yeast cells having an average size of about 7.5 microns and subjected to sound patterns at a frequency of 127 Hz, and Haematococcus algae cells having an average size of about 15 microns and subjected to sound patterns at a frequency of 94 Hz. As shown, optimized sound frequencies for a particular type of cell can be related to the size of the cell, such that higher frequencies can work better for smaller cells while lower frequencies can work better for larger cells. These results can be extrapolated to find or estimate optimized sound frequencies for other cell types based on their size.

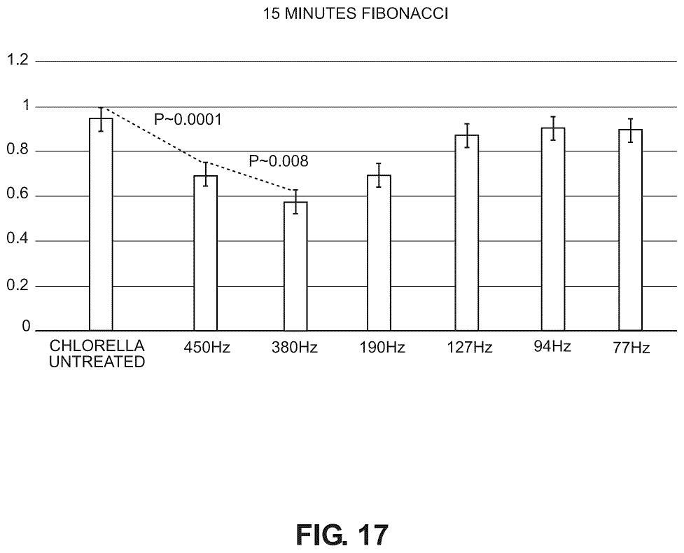

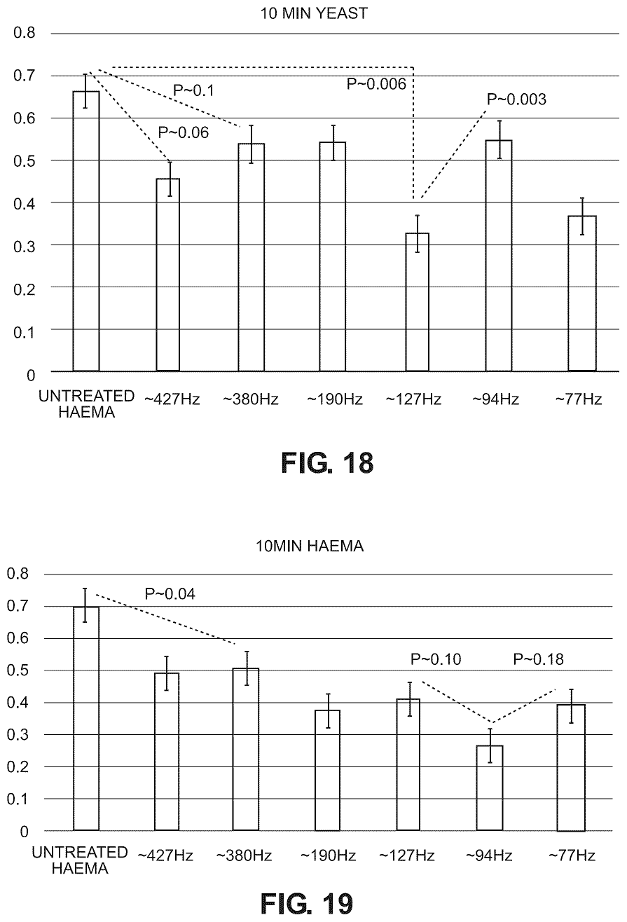

FIG. 17 illustrates charts of testing results on chlorella cells; FIG. 18 illustrates charts of testing results on yeast cells; FIG. 19 illustrates charts of testing results on Haematococcus algae cells. All of the results shown in FIGS. 17-19 reflect testing done on cells without nanoparticles using sound waves having Fibonacci sound patterns at different frequencies. The chlorella cells in FIG. 17 were subjected to sound waves for 15 minutes for each test at a different frequency as shown, while the yeast cells in FIG. 18 and the Haematococcus algae cells in FIG. 19 were subjected to sound waves for 10 minutes for each test at a different frequency as shown. The various testing results shown in FIGS. 17-19 confirm the results of similar tests performed with results shown in FIGS. 11A-11C above. In general, different frequencies of sound waves produce different results for each different type of cell when all other testing factors and parameters are held constant.

As confirmed in these test results, the frequency at which a wave pattern is imparted on a particular type of cell can have a significant impact on whether cells of that type are significantly harmed or not significantly harmed. As such, the frequency used for a particular cell treatment as disclosed herein can be tuned to target one type of cells, such as cancer cells, for example, while primarily leaving alone other types of cells in the same environment.

In various embodiments, one or more characteristics of the particles, nanoparticles, or functionalized nanoparticles used can be optimized to achieve a degree of resonance or other enhancement with respect to the type or types of cells being targeted. Where a specific type of cells being targeted have certain characteristics that distinguish those cells from other types of cells that should be largely unaffected by treatment, then the particles or nanoparticles used can be specifically selected, designed, or otherwise optimized based on the distinguishable characteristics of the cells being targeted. A given type of invasive cells being targeted may be more susceptible to resonance at certain wave frequencies or wave patterns than other cells that are desired to remain largely unaffected, and this can be enhanced by using the right type or types of nanoparticles or other particles.

For example, nanoparticles having a diameter of about 10 nm may induce resonance at certain wave frequencies and patterns for a type of invasive cells that are to be targeted, and that same size of nanoparticles may induce little to no resonance for other types of cells at the same wave frequencies and patterns. In other arrangements, nanoparticles having a diameter of about 50 nm may induce resonance at certain wave frequencies and patterns for a type of invasive cells that are to be targeted while leaving other cells alone. Of course, other particle sizes are also possible depending upon the materials used, the cell types being targeted, and the cell types being left alone.

The material compositions of the nanoparticles may also factor into the amount of resonance experienced with specific wave types, frequencies, and patterns for different types of cells. For example, gold nanoparticles of a first size may work well for a given sound pattern or other wave pattern, while silver nanoparticles of that same first size may not. Gold nanoparticles of a second size different than the first size may not work as well as the first size, while silver nanoparticles of that second size may work better. Surface features on the nanoparticles may also factor into improved levels of resonance for certain sound or wave patterns.

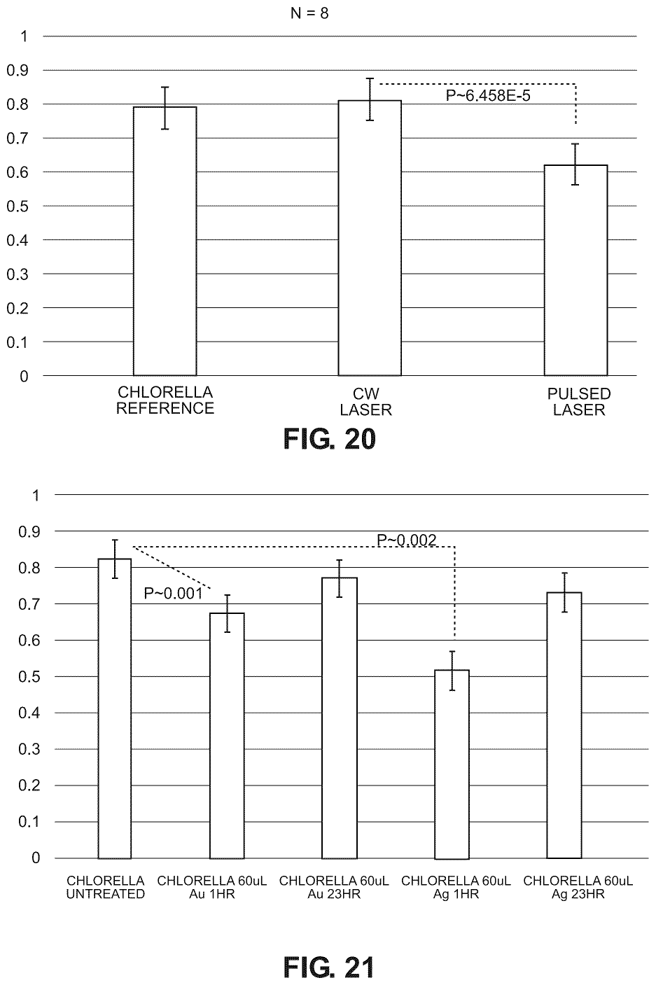

FIG. 20 illustrates charts of testing results on chlorella cells without nanoparticles using a continuous wave laser and a pulsed laser having a Fibonacci pulse pattern. In further testing arrangements, chlorella cell samples were subjected to light from a continuous wave laser and light from a pulsed laser. The continuous wave laser was a diode laser categorized as class IIIa, emitting less than 5 mW. The pulsed laser was operated at 638 nm at a frequency of 380 Hz for 12 minutes using a pulsed pattern having a Fibonacci sequence to the light pulses. Results of both laser tests compared to untreated chlorella reference cells are shown in FIG. 20. As shown, no significant difference was observed between the untreated references cells and the cells exposed to the continuous wave laser for a duration of 12 minutes. Conversely, a significant amount of dead cells was observed in the chlorella cell samples subjected to light from the pulsed laser when compared to both the untreated chlorella cell samples and the cell samples exposed to the continuous wave laser. The p-value in comparing cell samples exposed to the continuous wave laser to those that were treated with light from the pulsed laser in a pattern having a Fibonacci sequence was determined to be about 6.458E-5. Accordingly, it was demonstrated that light pulsed in a pattern having a Fibonacci sequence can be another type of wave that can be used to treat target cells. Such treatments can also be made using other types of wave patterns, such as magnetic waves and electromagnetic waves.

FIG. 21 illustrates charts of endocytosis testing and recovery results on chlorella cells using gold and silver nanoparticles. In further testing arrangements, different types and sizes of nanoparticles were introduced into different chlorella cell samples to determine the effects of endocytosis on the cells. In general, endocytosis is a cellular process where a cell takes up or ingests particles after a period of incubation, such as one hour, for example. Subject tests involved 8 different cell samples each for cells ingested with gold nanoparticles, cells ingested with silver nanoparticles, and untreated cells (i.e., control). The number of gold (Au) nanoparticles was 6E+7 at 60 uL, with a particle size of 10 nm, while the number of silver (Ag) nanoparticles was 6E+9 at 60 uL, with a particle size of 50 nm.

After one hour, the number of live to total cells in both the gold and silver samples decreased, with full recovery in both samples being experienced 23 hours later. Live cells decreased by 17% for those ingested with gold nanoparticles and decreased by 37% for those ingested with silver nanoparticles. The ingestion of nanoparticles caused death to the samples, albeit not complete. Interestingly, cells ingested with silver recovered at a higher level than those ingested with gold, despite the silver samples having orders of magnitude more particles than the gold samples, which can be due to the absorption peak spectrum correlating with the sizes of the nanoparticles. For gold, the absorption peak is at 552 nm at 54 nm in size; and for silver, the absorption peak is at 404 nm at 16 nm in size. Between the untreated samples and gold samples, there was a P-value of 0.001 and a Pearson correlation of 0.676. In comparison, between the untreated samples and silver samples, there was a slightly higher P-value of 0.002 and a Pearson correlation of 0.054.

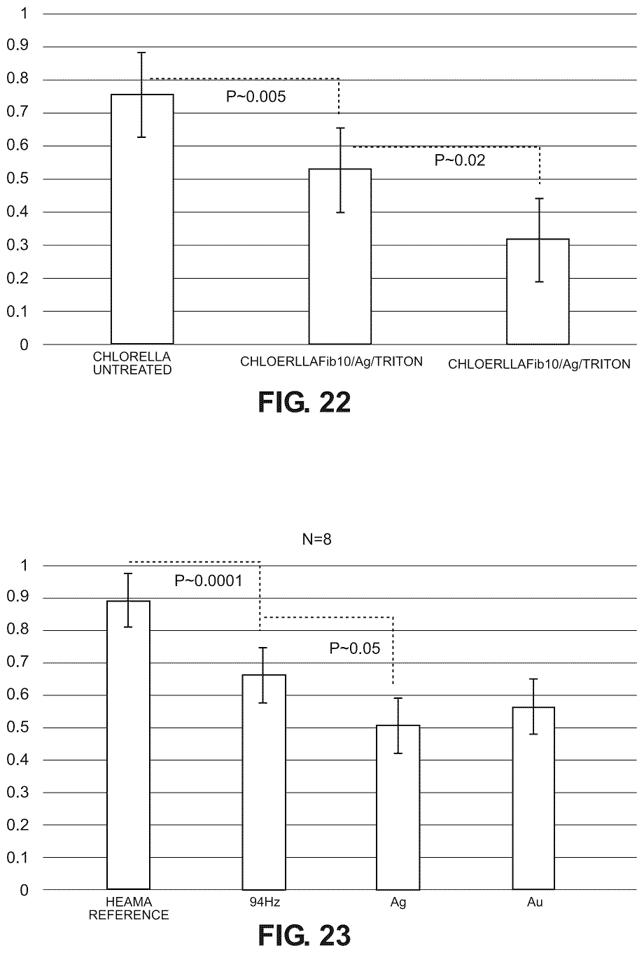

FIG. 22 illustrates charts of testing results on chlorella cells ingested with gold and silver nanoparticles and treated using sound waves having Fibonacci sound patterns at a set frequency, while FIG. 23 illustrates charts of testing results on Haematococcus algae cells ingested with gold and silver nanoparticles and treated using sound waves having Fibonacci sound patterns at a set frequency. After initial endocytosis tests, various cell samples were immediately subjected to sound patterns having a Fibonacci element. Chlorella cell samples were subjected to sound patterns at 190 Hz for 10 minutes, while Haematococcus algae cell samples were subjected to sound patterns at 94 Hz for 10 minutes.

In both of the chlorella and the Haematococcus algae cell samples, it was observed that introducing sound patterns having a Fibonacci element resulted in greater amounts of cell deaths than where no sound patterns were provided. For the chlorella cell samples subjected to Fibonacci sound patterns at 190 Hz, significantly more cell deaths were observed for cells that were ingested with gold nanoparticles than for cells that were ingested with silver nanoparticles. For the Haematococcus algae cell samples subjected to Fibonacci sound patterns at 94 Hz, however, significantly more cell deaths were observed for cells that were ingested with silver nanoparticles than for cells that were ingested with gold nanoparticles.

In addition to factors due to the differences in atomic and chemical makeups between the gold and silver nanoparticles, these differing results were also due to the size differences between the gold and silver nanoparticles (10 nm and 50 nm). These size differences also contributed to resonance differences for different sound wavelengths, such that the smaller gold nanoparticles were more effective than the larger silver nanoparticles in killing off chlorella cells using Fibonacci sound patterns at 190 Hz for 10 minutes, while the larger silver nanoparticles were more effective than the smaller gold nanoparticles in killing off Haematococcus algae cells using Fibonacci sound patterns at 94 Hz for 10 minutes.

As will be readily appreciated, these differing properties can all be varied and tuned for better cell damaging and destroying efficiencies based upon the specific cells being targeted and the neighboring cells that are to be preserved. For example, for a given type of invasive cell to be targeted for damage or destruction, a functionalized nanoparticle can be engineered to be formed from a specific material (e.g., gold, silver, titanium dioxide, iron oxide, liposome, dendrimer, etc.) that is a specific size (e.g., 5 nm, 10 nm, 20 nm, 50 nm, 100 nm, etc.) having a particular set of surface features (e.g., ligands, proteins, antibodies, geometric features, etc.) that all combine to resonate with waves at a specific frequency (e.g., sound waves at 190 Hz, sound waves at 94 Hz, light waves at 380 Hz, etc.), with those waves being delivered in a specific pattern (e.g., Fibonacci, arithmetic, or geometric sequence, etc.). Each of these specific variables can be tuned for targeting a specific type of cell, and ordinary experimentation and extrapolation can be used to optimize the right set of variables for damaging or destroying target cells while not damaging or destroying other neighboring cells that are different.

Cancer cells and other invasive agent cells often express specific molecules on their surfaces that other cells do not have. In some arrangements, these specific molecules can be identified as unique markers and can be specifically targeted. For example, specific ligands or other molecules can be used as targeting items to bind to these molecules specifically. These specific ligands or other molecules can be utilized as surface features on the functionalized nanoparticles or other particles that form part of the disclosed systems. A variety of features and techniques can be used form nanoparticles with such surface features.

In some arrangements, metal nanoparticles or other particles can be coated with targeting ligands that will predominately bind to a specific type of cancer cell or other invasive agent cell. Such metals can include, for example, gold, silver, and iron oxide, among other possible metals. In some arrangements, antibodies can be used to bind specific proteins onto the cancer cells or other targeted cells. Such antibodies can alternatively or additionally be coupled to surfaces of the nanoparticles such that the nanoparticles bind to target cells by way of the antibodies. Again, antibody surface features can be coated onto metal nanoparticles or other suitable particles, as noted above.

In addition to size considerations for inducing resonance or other enhanced effects with respect to targeted cells, it can also be preferable for the nanoparticles used to be small enough to effectively penetrate living tissue and cells. Modifying the surface charges of functionalized nanoparticles can also enhance the interactions between the particles and the cancer cells or other invasive agent cells being targeted. For some nanoparticles that have a metallic component, such as iron oxide, for example, one or more magnetic fields can be used to help guide the nanoparticles through the human or other living being, such as to a tumor site.

In various embodiments, metal nanoparticles or other particles being used within a human or other living being can be engineered to be safe for internal use, such that poisoning or adverse reactions do not occur. This can involve coating the nanoparticles with a suitable amount and type of biocompatible materials. Such materials can be used in a manner that offsets the effects of the base nanoparticles as well as any ligands or other surface features.

In some arrangements, the composition of the serum or other delivery fluid or medium can be specifically designed to enhance the delivery of sound vibrations or other waves or vibrations to targeted cells. For example, a serum or other fluid containing functionalized nanoparticles or other cell targeting particles can include specialized proteins, other metals, and/or other materials that can facilitate the delivery of sound vibrations, other vibrations, light waves, magnetic waves, or other treatment waves through the living being and to the cells being targeted. Other enhancing properties are also possible.

It will be appreciated that the present disclosure may include any one and up to all of the following examples.

Example 1. A system configured to treat invasive agent cells, the system comprising: a plurality of particles configured for introduction into a living being, wherein the plurality of particles includes surface features configured to facilitate attachment of at least a portion of the plurality of particles to invasive agent cells within the living being; and a wave generation system configured to provide a specific pattern of waves to the living being, the specific wave pattern including waves at one or more frequencies, wherein the specific wave pattern is configured to actuate the plurality of particles to damage, destroy, or create an immune response to the invasive agent cells to which they are attached.

Example 2. The system of Example 1, wherein the living being is a human and the invasive agent cells are cancer cells.

Example 3. The system of any one of Examples 1-2, wherein the plurality of particles includes functionalized nanoparticles selected from the group consisting of gold, silver, liposomes, and dendrimers.

Example 4. The system of any one of Examples 1-3, wherein the plurality of particles is contained within a fluid configured for injection into the bloodstream of the living being.

Example 5. The system of any one of Examples 1-4, wherein actuating the plurality of particles includes inducing one or more movements, rotations, or changes in temperature, shape, confirmation, or chemical structure in at least a portion of the plurality of particles in response to the specific wave pattern.

Example 6. The system of any one of Examples 1-5, wherein actuating the plurality of particles results in lysis of or immune response to invasive agent cells to which the at least a portion of the plurality of particles are attached.

Example 7. The system of any one of Examples 1-6, wherein the specific wave pattern includes pulses of waves interspersed between pulses of no waves, and wherein durations of the pulses vary to form ratios between the pulses of waves and the pulses of no waves.

Example 8. The system of Example 7, wherein the relationship of the ratios forms an arithmetic, geometric, or Fibonacci sequence.

Example 9. The system of any one of Examples 1-8, wherein the waves include sound waves.

Example 10. The system of any one of Examples 1-9, wherein the waves include light waves, magnetic waves, or electromagnetic waves.

Example 11. A method of treating an invasive agent within a living being, the method comprising: providing a plurality of particles, wherein the plurality of particles includes surface features configured to facilitate attachment of a significant amount of the plurality of particles to invasive agent cells within the living being; introducing the plurality of particles into the living being; facilitating attachment of a significant amount of the plurality of particles to invasive agent cells within the living being; and directing a specific wave pattern toward the living being, the specific wave pattern including waves at one or more frequencies, wherein the specific wave pattern is configured to actuate the plurality of particles to damage, destroy, or create an immune response to the invasive agent cells to which they are attached.

Example 12. The method of Example 11, wherein the specific wave pattern includes pulses of waves interspersed between pulses of no waves, and wherein durations of the pulses vary to form ratios between the pulses of waves and the pulses of no waves.

Example 13. The method of Example 12, wherein the relationship of the ratios forms an arithmetic, geometric, or Fibonacci sequence.

Example 14. The method of any one of Examples 11-13, further comprising the step of: actuating the plurality of particles automatically with the specific wave pattern, wherein the actuating results in damaging, destroying, or provoking an immunological response to invasive agent cells to which any of the plurality of particles are attached.

Example 15. The method of Example 14, wherein actuating the plurality of particles involves rotational or translational movement of the particles due to the specific wave pattern.

Example 16. The method of any one of Examples 11-15, wherein the waves include sound waves and wherein the plurality of particles include functionalized nanoparticles.

Example 17. A system configured to treat living cells, the system comprising: a wave generation system configured to project a specific wave pattern, the specific wave pattern including waves at one or more frequencies, wherein the specific wave pattern is configured to induce lysis of or immune response to a significant amount of living cells within a living being.

Example 18. The system of Example 17, wherein the specific wave pattern includes pulses of waves interspersed between pulses of no waves, wherein durations of the pulses vary to form ratios between the pulses of waves and the pulses of no waves, and wherein the relationship of the ratios of the pulses forms an arithmetic, geometric, or Fibonacci sequence.

Example 19. The system of Example 18, wherein the specific wave pattern includes a specific sound pattern having a Fibonacci word element, wherein the Fibonacci word element includes a series of 0 and 1 values that each represent pulses of no sound and pulses of sound respectively, and wherein each of the 0 and 1 pulses have the same time length.

Example 20. The system of any one of Examples 17-19, further comprising: a plurality of functionalized nanoparticles configured for introduction into the living being, wherein the plurality of functionalized nanoparticles includes surface features configured to facilitate attachment of the functionalized nanoparticles to living cells within the living being without also facilitating attachment of the functionalized nanoparticles to a significant amount of healthy cells of the living being, and wherein the functionalized nanoparticles are configured to be actuated by the specific wave pattern.

Example 21. A system configured for treating invasive agent cells, the system comprising: a plurality of nanoparticles configured for introduction into a living being, wherein the plurality of nanoparticles includes surface features configured to facilitate attachment of at least a portion of the plurality of nanoparticles to invasive agent cells within the living being without also facilitating attachment of a significant amount of the plurality of nanoparticles to healthy cells of the living being; and a sound generation system configured to provide a specific sound pattern to the living being, the specific sound pattern including sound waves at one or more sound frequencies, wherein the specific sound pattern is configured to actuate the plurality of nanoparticles to damage, destroy, or create an immune response to the invasive agent cells to which they are attached.

Example 22. The system of Example 21, wherein the living being is a human and the invasive agent cells are cancer cells.

Example 23. The system of any one of Examples 21-22, wherein the plurality of nanoparticles includes functionalized nanoparticles selected from the group consisting of gold, titanium dioxide, silver, liposomes, and dendrimers.

Example 24. The system of any one of Examples 21-23, wherein the plurality of nanoparticles is contained within a fluid configured for injection into the bloodstream of the living being.

Example 25. The system of any one of Examples 21-24, wherein the nanoparticle surface features include one or more citrate, lactate, glycol, or norbornene components, polymeric strings having a multiple of such components in a repeating pattern, or any combination thereof.

Example 26. The system of any one of Examples 21-25, wherein the nanoparticle surface features include one or more biological components.

Example 27. The system of any one of Examples 21-26, wherein the one or more biological components include one or more antibodies or other proteins.

Example 28. The system of any one of Examples 21-27, wherein actuating the plurality of nanoparticles includes inducing one or more movements, rotations, or changes in temperature, shape, confirmation, or chemical structure in each of at least a portion of the plurality of nanoparticles in response to the specific sound pattern.

Example 29. The system of Example 28, wherein the actuating results in lysis of or immune response to invasive agent cells to which the at least a portion of the plurality of nanoparticles are attached.

Example 30. The system of any one of Examples 21-29, wherein the specific sound pattern includes pulses of infrasound or ultrasound interspersed between pulses of no sound, sound at audible or non-audible frequencies, or both, and wherein the lengths of the pulses of infrasound or ultrasound vary to form ratios between the pulses of infrasound or ultrasound.

Example 31. The system of any one of Examples 21-30, wherein the relationship of the ratios of the pulses of sound forms an arithmetic, geometric, or Fibonacci sequence.

Example 32. A method of treating an invasive agent within a living being, the method comprising: providing a plurality of nanoparticles, wherein the plurality of nanoparticles includes surface features configured to facilitate attachment of a significant amount of the plurality of nanoparticles to invasive agent cells within the living being without also facilitating attachment of a significant amount of the plurality of nanoparticles to healthy cells of the living being; introducing the plurality of nanoparticles into the living being; facilitating attachment of a significant amount of the plurality of nanoparticles to invasive agent cells within the living being; and directing a specific sound pattern toward the living being, the specific sound pattern including sound waves at infrasound or ultrasound frequencies, wherein the specific sound pattern is configured to actuate the plurality of nanoparticles to damage or destroy invasive agent cells to which they are attached.

Example 33. The method of Example 32, wherein the facilitating attachment includes allowing a sufficient amount of time for the significant amount of the plurality of nanoparticles to attach to the invasive agent cells.

Example 34. The method of any one of Examples 32-33, wherein the introducing involves injecting into the living being a fluid containing the plurality of nanoparticles.

Example 35. The method of any one of Examples 32-34, wherein the specific sound pattern includes pulses of infrasound or ultrasound interspersed between pulses of no sound, sound at audible or non-audible frequencies, or both, and wherein the lengths of the pulses of sound vary to form ratios between the pulses of sound.

Example 36. The method of any one of Examples 32-35, wherein the relationship of the ratios of the pulses of sound forms an arithmetic, geometric, or Fibonacci sequence.

Example 37. The method of any one of Examples 32-36, further comprising the step of: actuating the plurality of nanoparticles automatically with the specific sound pattern, wherein the actuating results in damaging, destroying, or provoking an immunological response to invasive agent cells to which any of the plurality of nanoparticles are attached.

Example 38. The method of Example 37, wherein actuating the plurality of nanoparticles involves rotational or translational movement of the nanoparticles due to the specific sound pattern.

Example 39. A system configured for treating cancer within a human, the system comprising: a sound generation system configured to project a specific sound pattern at the human, the specific sound pattern including sound waves at infrasound or ultrasound frequencies, wherein the specific sound pattern is configured to induce lysis of or immune response to a significant amount of cancer cells within the human.

Example 40. The system of Example 39, wherein the specific sound pattern includes pulses of sound interspersed between pulses of no sound, and wherein the lengths of the pulses of sound vary to form ratios between the pulses of sound.

Example 41. The system of Example 40, wherein the relationship of the ratios of the pulses of sound forms an arithmetic, geometric, or Fibonacci sequence.

Example 42. The system of any one of Examples 39-41, wherein the specific sound pattern includes a Fibonacci word element within the specific sound pattern.

Example 43. The system of Example 42, wherein the Fibonacci word element includes a series of 0 and 1 values that each represent pulses of no sound and pulses of sound respectively.

Example 44. The system of Example 43, wherein each of the 0 and 1 pulses have the same time length.

Example 45. The system of any one of Examples 42-44, wherein the Fibonacci word element within the specific sound pattern is the overwhelming or only cause of inducing the lysis of or the immune response to the significant amount of cancer cells.

Example 46. The system of any one of Examples 39-45, wherein the specific sound pattern does not cause any significant lysis of or immune response to any other cells within the human besides the cancer cells.

Example 47. The system of any one of Examples 39-46, further comprising: a plurality of functionalized nanoparticles configured for introduction into the human, wherein the plurality of functionalized nanoparticles includes surface features configured to facilitate attachment of the functionalized nanoparticles to cancer cells within the human without also facilitating attachment of the functionalized nanoparticles to a significant amount of healthy cells of the living being, and wherein the functionalized nanoparticles are configured to be actuated by the specific sound pattern.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims.

What is claimed is:

1. A system configured to treat invasive agent cells, the system comprising:
a plurality of particles configured for introduction into a living being, wherein the plurality of particles includes surface features configured to facilitate attachment of at least a portion of the plurality of particles to invasive agent cells within the living being; and
a wave generation system configured to provide a specific pattern of waves to the living being, the specific wave pattern including waves at one or more frequencies, wherein the specific wave pattern is configured to actuate the plurality of particles to damage, destroy, or create an immune response to the invasive agent cells to which they are attached.

2. The system of claim 1, wherein the living being is a human and the invasive agent cells are cancer cells.

3. The system of claim 1, wherein the plurality of particles includes functionalized nanoparticles selected from the group consisting of gold, silver, liposomes, and dendrimers.

4. The system of claim 1, wherein the plurality of particles is contained within a fluid configured for injection into the bloodstream of the living being.

5. The system of claim 1, wherein actuating the plurality of particles includes inducing one or more movements, rotations, or changes in temperature, shape, confirmation, or chemical structure in at least a portion of the plurality of particles in response to the specific wave pattern.

6. The system of claim 1, wherein actuating the plurality of particles results in lysis of or immune response to invasive agent cells to which the at least a portion of the plurality of particles are attached.

7. The system of claim 1, wherein the specific wave pattern includes pulses of waves interspersed between pulses of no waves, and wherein durations of the pulses vary to form ratios between the pulses of waves and the pulses of no waves.

8. The system of claim 7, wherein the relationship of the ratios forms an arithmetic, geometric, or Fibonacci sequence.

9. The system of claim 1, wherein the waves include sound waves.

10. The system of claim 1, wherein the waves include light waves, magnetic waves, or electromagnetic waves.

11. A method of treating an invasive agent within a living being, the method comprising:
providing a plurality of particles, wherein the plurality of particles includes surface features configured to facilitate attachment of a significant amount of the plurality of particles to invasive agent cells within the living being;
introducing the plurality of particles into the living being;
facilitating attachment of a significant amount of the plurality of particles to invasive agent cells within the living being; and
directing a specific wave pattern toward the living being, the specific wave pattern including waves at one or more frequencies, wherein the specific wave pattern is configured to actuate the plurality of particles to damage, destroy, or create an immune response to the invasive agent cells to which they are attached.

12. The method of claim 11, wherein the specific wave pattern includes pulses of waves interspersed between pulses of no waves, and wherein durations of the pulses vary to form ratios between the pulses of waves and the pulses of no waves.

13. The method of claim 12, wherein the relationship of the ratios forms an arithmetic, geometric, or Fibonacci sequence.

14. The method of claim 11, further comprising the steps of:
actuating the plurality of particles automatically with the specific wave pattern, wherein the actuating results in damaging, destroying, or provoking an immunological response to invasive agent cells to which any of the plurality of particles are attached.

15. The method of claim 14, wherein actuating the plurality of particles involves rotational or translational movement of the particles due to the specific wave pattern.

16. The method of claim 11, wherein the waves include sound waves and wherein the plurality of particles include functionalized nanoparticles.

17. A system configured to treat invasive cells, the system comprising:
a wave generation system configured to project a specific wave pattern, the specific wave pattern including waves at one or more frequencies, wherein the specific wave pattern is configured to induce lysis of or immune response to a significant amount of invasive cells within a living being.

18. The system of claim 17, wherein the specific wave pattern includes pulses of waves interspersed between pulses of no waves, wherein durations of the pulses vary to form ratios between the pulses of waves and the pulses of no waves, and wherein the relationship of the ratios of the pulses forms an arithmetic, geometric, or Fibonacci sequence.

19. The system of claim 18, wherein the specific wave pattern includes a specific sound pattern having a Fibonacci word element, wherein the Fibonacci word element includes a series of 0 and 1 values that each represent pulses of no sound and pulses of sound respectively, and wherein each of the 0 and 1 pulses have the same time length.

20. The system of claim 17, further comprising:
a plurality of functionalized nanoparticles configured for introduction into the living being, wherein the plurality of functionalized nanoparticles includes surface features configured to facilitate attachment of the functionalized nanoparticles to the invasive cells within the living being without also facilitating attachment of the functionalized nanoparticles to a significant amount of healthy cells of the living being, and wherein the functionalized nanoparticles are configured to be actuated by the specific wave pattern.
